(19) United States
(12) Patent Application Publication
(10) Pub. No.: US 2025/0040830 A1
(43) Pub. Date: Feb. 6, 2025

(54) SYSTEM AND METHOD FOR MONITORING A PHYSIOLOGICAL STATE OF A USER AND PROVIDING AT LEAST ONE PERSONALIZED BREATHING EXERCISE TO THE USER, AND VIRTUAL MONITORING PROGRAM FOR EXECUTING THE METHOD

(71) Applicant: NANOLEQ AG, Rümlang (CH)

(72) Inventors: Serge WEYDERT, Zurich (CH); Vincent MARTINEZ, Zurich (CH); Flurin STAUFFER, Zurich (CH); Niclas GRANQVIST, Mägenwil (CH); Masih NILCHIAN, Zürich (CH); Ali Fahri ANDER, Zürich (CH)

(21) Appl. No.: 18/706,874

(22) PCT Filed: Nov. 2, 2021
(86) PCT No.: PCT/EP2021/080420

§ 371 Date: May 2, 2024

(51) Int. Cl. A61B 5/08; A61B 5/00; A61B 5/113

(52) U.S. Cl. CPC A61B 5/0809; A61B 5/113; A61B 5/4848; A61B 5/486; A61B 5/6804; A61B 5/7475

(57) ABSTRACT

The present invention relates to a system (10) for monitoring a physiological state and/or a mental state and/or an emotional state of a user and providing a personalized breathing exercise to the user, the system including a physiological monitoring device (12) configured to detect a physiological parameter of the user and a breathing monitoring device (14) configured to detect a breathing parameter of the user. The system (10) is configured to determine a target physiological index based at least on the detected physiological parameter, determine a target breathing exercise to be executed by the user based at least on the target physiological index, determine a target breathing index to be achieved by the user during execution of the breathing exercise, compare the target breathing index with the breathing parameter, which is detectable as the user is executing the breathing exercise, and determine a deviation therebetween, and adapt the target breathing exercise based at least on the determined deviation. The invention also relates to a method and a monitoring program for executing the method.

Monitoring the physical and/or psychological condition of humans is essential for ensuring a general well-being. For instance, checking cardiovascular parameters, e.g., a heart rate, heart rate variability and a blood pressure, on a regular basis can provide a relatively reliable indication of the general physical and/or psychological condition of a person.

In recent years, the availability and use of wearable devices for monitoring physiological parameters of users for recreational use have become widespread. Wearable devices offer advantages versus devices which are generally used for measurements in a stationary setting, such as when visiting a medical practitioner for a medical checkup, e.g., since wearable devices may allow the user's general well-being to be assessed more practically in real situations during a user's daily life. In addition, wearable devices may promote a more proactive involvement of the respective user in monitoring and managing factors, such as stress, which may have an effect on the user's well-being, in particular due to the integration of the monitoring and managing of such factors into the user's daily life.

Smart garments are generally worn relatively closely to vital organs of the user such that vital signals, such as heart rate (HR) and heart rate variability (HRV), may be measured in an unobtrusive and continuous way. Thus, the smart garments may be worn comfortably for longer periods of time, e.g., throughout the day and/or night, which makes smart garments particularly useful in recreational applications, such as for general well-being and lifestyle monitoring during leisure activities and/or sports, and/or during manual labor, e.g., during job-related manual labor, such as construction work.

Some monitoring devices provide feedback and/or advice to the user for positively affecting the monitored parameters. For instance, some devices may suggest measures, such as a physical activity, e.g., an exercise, which are aimed at positively affecting the user's general well-being.

However, disadvantages still remain in the devices known from the prior art. For instance, the known devices do not ensure, or at least provide sufficient means to check, that the suggested measures are effective in positively affecting the user's general well-being. Moreover, the known devices do not provide further measures to increase the effectiveness of the originally suggested measures in case the originally suggested measures are not effective, or not as effective as desired. Besides, known devices do not check and ensure if the suggested measures are actually conducted and if they are conducted in the correct manner.

It is therefore an object of the present invention to provide enhanced means for assessing and positively affecting a user's well-being.

This object is achieved by a system defined by the features of claim 1. Variations and further developments are defined by the features of the dependent claims.

Accordingly, the present invention relates to a system for monitoring a physiological state of a user and providing at least one personalized breathing exercise to the user. The system includes at least one physiological monitoring device configured to detect at least one physiological parameter of the user and at least one breathing monitoring device configured to detect at least one breathing parameter of the user. The system is configured to determine at least one target physiological index based at least on the detected physiological parameter and to determine a target breathing exercise to be executed by the user based at least on the target physiological index. The system is further configured to determine a target breathing index to be achieved by the user during execution of the breathing exercise and compare the target breathing index with the breathing parameter, which is detectable as the user is executing the breathing exercise, and determine a deviation therebetween. The system is further configured to adapt the target breathing exercise based at least on the determined deviation.

The physiological monitoring device and/or the breathing monitoring device, respectively, may be configured as a wearable device which may include means for attaching to the user's body and/or to be integrated into a garment, preferably a garment configured to be worn on the upper body of the user, e.g., a shirt or a belt. Preferably, the physiological monitoring device and/or the breathing monitoring device, respectively, are configured as mobile devices such that the user can wear the physiological monitoring device and/or the breathing monitoring device and remain mobile meanwhile.

The physiological parameter may be a cardiovascular parameter, such as a heart rate, a heart rate variability, a systolic and/or diastolic blood pressure, a blood pulse volume, and/or a blood oxygen level. In particular, the physiological parameter may be detected via an electrical current generated by the user's heart, e.g., an electrocardiographic (ECG/EKG) signal.

Alternatively, the cardiovascular parameter may be detected via photoplethysmography (PPG) and/or pulse oximetry. Alternatively, or additionally, the physiological parameter may be a parameter related to a skin conductance of the user. In particular, a change in electrical conductance of the skin may occur in response to sweating by the user. Thus, detecting the change in the electrical conductance of the user's skin, and thus the presence of sweating and/or a severity of sweating by the user, may give a general indication of the user's physical state and/or psychological state and/or general well-being.

The physiological monitoring device may be configured to detect an electrodermal activity (EDA) parameter or a galvanic skin response (GSR) parameter to determine the user's skin conductance and/or a change in the skin conductance. Alternatively, or additionally, the physiological parameter may be a skin impedance, which may be detectable by using electrochemical impedance spectroscopy. Alternatively, or additionally, the physiological parameter may be based on detecting the presence and/or an amount and/or a concentration of at least one component, preferably a plurality of components, in the user's sweat. In particular, the component may be one or more types of ions, recreational drugs and/or medicinal drugs, metabolites, biomolecules, hormones or any other component(s) which may occur in the sweat of a human. Alternatively, or additionally, the physiological parameter may be an estimation of the general physical, mental and/or emotional state of the user, such as a level of stress, e.g., physical, psychological and/or oxidative stress, relaxation, fatigue, concentration, focus, surprise, happiness, depression, anxiety, excitement or other emotional state. For the purpose of detecting the physiological parameter, the physiological monitoring device may include one or more detecting elements, such as one or more electrodes, configured to detect the physiological parameter. The physiological monitoring device may be configured to detect a plurality of physiological parameters at least partially simultaneously and/or in a time-shifted manner. Alternatively, or additionally, the physiological parameter may be a muscle activity, e.g., which may be detectable by means of electromyography (EMG), and/or a general physical activity of the user, e.g. a jumping, running or walking movement, which may be detectable by means of an accelerometer, a gyroscope and/or a magnetometer. Alternatively, or additionally, the physiological parameter may be a neuronal activity and/or a brain activity, e.g., which may be detectable by means of electroencephalography (EEG). Alternatively, or additionally, the physiological parameter may be a CO2 level and/or a partial pressure of CO2 in the blood and/or in the ventilated air of the user, e.g., which may be detectable by means of capnography. Alternatively, or additionally, the physiological parameter may be a CO2 tolerance and/or a CO2 sensitivity, e.g., which may be estimated by means of a Control Pause test or a Body Oxygen Level Test (BOLT).

The target physiological index may be a certain physiological value of a specific physiological parameter, e.g., a specific value of a heart rate or a heart rate variability, and/or a minimum value and/or a maximum value. Additionally, or alternatively, the target physiological index may be a certain range of values, such as a range defined by a minimum value and a maximum value. The target physiological index may also be a combination of one or more values and/or one more ranged of values of one or more physiological parameters. Additionally, or alternatively, the target physiological index may be just a general direction of change of at least one physiological parameter of the user, e.g., a general reduction in the heart rate or an increase in heart rate variability of the user.

The target physiological index may be represented by a sequence of physiological feature vectors. The sequence may be configured as a time series. Each physiological feature vector may be an N-dimensional numerical vector, wherein N>=1 and N may be defined for a window, wherein the window may be a time window at a specific time point or a window with a size delta_t>0 and/or a window over an exercise phase. For example, the target physiological index at a given time t may be denoted by a target physiological vector TPV (t), and a sequence of breathing feature vectors for a given time sequence {t_0, t_1, . . . , t_n} may be denoted by TPV^{(t_0, t_n)}. The elements of this vector may include, but are not limited to, one or more of the physiological parameters as described above, such as:
a heart rate, a heart rate variability, a blood pressure, an arterial stiffness, an arterial elasticity, a pulse wave velocity, a blood oxygen level, a CO2 tolerance, a CO2 level, a skin impedance, a skin conductance, and/or a stress level etc. (list 1);
a linear and/or nonlinear transformation of any of the presented features in list 1 (list 2);
a variability of any of the presented features in list 1 and list 2 (list 3);
a statistic of any of features presented in list 1 and/or list 2 and/or list 3 (list 4); and/or
any combination of the features presented in list 1, list 2, list 3 and/or list 4.

The statistic may be selected from, but is not limited to, a mean, a median, a variance, a standard deviation, and an entropy. The variability may be selected from, but is not limited to, a mean, a median, a variance, a standard deviation, an entropy, a root mean square of sequential difference, and a Fourier power spectrum.

The breathing parameter(s) may include any of the following: a breathing frequency (respiratory rate), a breathing volume, an inhale volume, an exhale volume, an inspiratory time, an expiratory time, a breath pause time, a respiratory duty cycle, a total breath time, a breathing pattern and/or any other breathing parameter which may be determined based on any of these parameters. For the purpose of detecting the breathing parameter, the breathing monitoring device may be configured to detect the breathing parameter by means of one or more sensing elements, preferably tactile sensing elements, configured to sense movement of the user's chest, e.g., by detecting an inflation and/or deflation of the user's chest, as the user is breathing to detect the breathing parameter. The breathing monitoring device may be configured to tactically measure the breathing parameter, e.g., by tactically measuring an expansion of the user's chest, preferably by means of at least one strain gauge, e.g., a plethysmography belt which is wearable around the user's chest. Alternatively, or additionally, the breathing monitoring device may be configured to optically detect the breathing parameter, e.g., by optically detecting movement, e.g., an inflation and/or deflation, of the user's chest as the user is breathing, e.g., preferably by means of optoelectronic plethysmography. For this purpose, the breathing monitoring device may include one or more optical sensors, such as a camera sensor, configured to optically detect the breathing parameter. Alternatively, or additionally, the breathing monitoring device may be configured to acoustically detect the breathing parameter by a sound sensing device, such as a microphone or an array or matrix of microphones. Alternatively, or additionally, the breathing monitoring device may be configured to measure the breathing parameter by a temperature sensor, a flow sensor and/or a pressure sensor which may be mountable in a flow path of the user's breathing, e.g., on or near the nose or mouth of the user. Alternatively, or additionally, the breathing monitoring device may be configured to detect the presence and/or concentration of a component in a human's expiration, such as carbon dioxide, oxygen, nitrogen, water vapor or humidity, and/or argon, in order to detect the breathing parameter.

The target breathing index may be related to any type of breathing characteristic which is at least partially controllable by the user during breathing and which may be determined by the breathing monitoring device as the user is executing the breathing exercise. The target breathing index may be a breathing frequency (respiratory rate), a breathing volume, an inhale volume, an exhale volume, an inspiratory time, an expiratory time, a respiratory duty cycle, a total breath time, a breath pause time, a breathing pattern or any other breathing parameter which may be determined based on any of these parameters. The breathing monitoring device may be configured to detect a plurality of breathing parameters at least partially simultaneously and/or in a time-shifted manner.

The target breathing index may be a certain value, such as a certain breathing frequency (respiratory rate) value and/or a minimum value and/or a maximum value. Additionally, or alternatively, the target breathing index may be a certain range of values, such as a range defined by a minimum value and a maximum value. Additionally, or alternatively, target breathing index may be just a general direction of change of at least one breathing characteristic of the user's breathing, e.g., a general reduction in the breathing frequency (respiratory rate) of the user's breathing. Alternatively, or additionally, the target breathing index may be a manner in which the user breathes in general, such as diaphragmatic, deep, eupneic, costal, shallow breathing, hyperpnea, hyperventilation, hypoventilation or a breath pause/apnea.

The target breathing index may be represented as a sequence of feature vectors. The sequence may be configured as a time series. Each breathing feature vector may be an N-dimensional numerical vector, wherein N>=1 and N may be defined for each breath or for each sequence of a plurality of breaths. For example, the target breathing feature vector at a given time t may be denoted by TBV^{(t)}, and a sequence of breathing feature vectors for a given time sequence {t_0, t_1, . . . , t_n} may be denoted by TBV^{(t_0, t_n)}. The elements of this vector may include, but are not limited to, the following:
breathing rate, breathing volume, inhale time, exhale time, breath pause time, time difference between two consecutive breaths, breathing signal values (list 5);
a linear and/or nonlinear transformation of any of the presented features in list 5 (list 6);
a variability of any of the presented features in list 5 and/or list 6 (list 7);
a statistic of any of the features presented in list 5 and/or list 6 and/or list 7 (list 8); and/or
any combination of the features presented in list 5, list 6, list 7, list 8.

The statistic may be selected from, but is not limited to, a mean, a median, a variance, a standard deviation, and an entropy. The variability may be selected from, but is not limited to, a mean, a median, a variance, a standard deviation, an entropy, a root mean square of sequential difference, and a Fourier power spectrum.

The system may be configured to determine the target physiological index substantially in real-time based on a physiological parameter which is being detected as the target physiological index is being determined. Alternatively, or additionally, the system may be configured to determine the target physiological index based on a detected physiological parameter which was measured sometime in the past and was stored in a database comprising historical detection data and which is accessible by the system. The system may be configured such that the target physiological index is determined based not only on the detected physiological parameter. For instance, the target physiological index may be determined based on further data and/or criteria, such as on user input, stored data, such as historical measurement data, such as parameters which were detected in the past, and/or an effectiveness of one or more specific breathing exercises and/or the general effectiveness of breathing exercises in improving the physiological state of the user and/or a compliance of the user with respect to of one or more specific breathing exercises, the historical measurement data being accessible by the system.

Preferably, the system may be configured to indicate the target breathing exercise, and optionally also the actual executed breathing exercise, visually and/or acoustically to the user, preferably in a step-by-step manner. In case the system is configured to indicate the target breathing exercise, and optionally also the actual executed breathing exercise, visually to the user, the system may be configured to provide an animation of the target breathing exercise, and optionally also the actual executed breathing exercise, to the user. In particular, the system may be configured to provide a comparison between the target breathing exercise and the actual executed breathing exercise, e.g., so that the user can adjust the execution of the breathing exercise to match, or at least more closely align with, the target breathing exercise. For the purpose of indicating the target breathing exercise, and optionally also the actual executed breathing exercise, to the user, the system may include a display configured to visually display the target breathing exercise, and optionally also the actual executed breathing exercise, to the user and/or means, e.g., at least one speaker and/or outlet for providing at least one speaker signal, for acoustically indicating the target breathing exercise, and optionally also the actual executed breathing exercise, to the user.

The target breathing exercise may be determined by individually determining the target breathing exercise on a user to user basis, e.g., by means of at least one algorithm which is configured to determine the target breathing exercise based at least on the target physiological index. Alternatively, the target breathing exercise may be selected from a variety of predetermined target breathing exercises, which may be accessible from a storage, based at least on the target physiological index.

Adapting the target breathing exercise may occur substantially in real-time as the user is executing the breathing exercise, and/or in a time-shifted manner, e.g., after the execution of the breathing exercise has been completed. “Adapt” may mean that one or more aspects or parameters of the breathing exercise are changed, e.g., that a duration and/or frequency and/or an intensity and/or a speed of one or more breathing phases may be changed.

The system described herein may provide a reliable means for generally assessing the user's general well-being by detecting at least one physiological parameter of the user by means of the physiological monitoring device. By determining at least one target physiological index based at least on the detected physiological parameter, the system may provide a target for improving the physical and/or psychological state of the user.

In addition, determining a target breathing exercise to be executed by the user based at least on the target physiological index and determining a target breathing index to be achieved by the user during execution of the breathing exercise may provide a means for improving the user's physiological state and/or mental state and/or emotional state based on the detected physiological parameter by guiding and/or altering the user's breathing via the target breathing exercise and the target breathing index. In fact, the way humans breathe may influence their physical, mental and emotional well-being and may have significant effects both on the cardiovascular and the autonomic nervous system. For example, slow breathing may have significant effects on the respiratory, cardiovascular, cardiorespiratory and autonomic nervous systems, including effects on respiratory muscle activity, ventilation efficiency, chemoreflex and baroreflex sensitivity, heart rate variability, blood flow dynamics, respiratory sinus arrhythmia, cardiorespiratory coupling, and sympathovagal balance, as described in the scientific article “The physiological effects of slow breathing in the healthy human” by Marc. A Russo et al. (Breathe13, pages 298-309, DOI: 10.1183/20734735.009817, December 2017), the content of which is herewith incorporated by reference in its entirety. Thus, by changing breathing habits or performing breathing exercises, the autonomous nervous system may be influenced to positively impact the general health and well-being of the user. For example, slow breathing may enhance the activity of the parasympathetic nervous system, resulting in an increase in comfort and relaxation while reducing symptoms such as stress, anxiety or depression. Furthermore, breathing exercises may positively impact the quality of life on a wide variety of users suffering from diseases such as asthma, cancer, eating disorders, hypertension, migraine, anxiety, sleep apnea, gastroesophageal reflux disease and cardiovascular conditions. Also, breathing exercises may be used to activate the sympathetic nervous system, for example by hyperventilation and/or elongated breath holds. This may influence the innate immune response and change hormone release (such as cortisol or adrenaline) in the body through a voluntary action. Furthermore, such breathing exercises may be used to perform hypoxic training or train the tolerance of air hunger. Hence, the system described herein may improve the user's physical, mental and emotional well-being by providing guidance to the user's breathing by suggesting various breathing exercises.

In addition, by being able to monitor the user's breathing by the breathing monitoring device during execution of the breathing exercise, compare the target breathing index with the breathing parameter detected as the user is executing the breathing exercise, and determine a deviation therebetween by the system, an effective and/or efficient and/or comfortable and/or compliant execution of the target breathing exercise may be monitored to determine whether the execution of the breathing exercise may be optimized in order provide a higher effectiveness of the breathing exercise in improving, or at least mitigating negative effects on, the user's physical, mental and emotional well-being. The way individual people typically breathe and their actual breathing capabilities may differ from one individual to the next. Thus, based on the determined deviation between the target breathing index and the breathing parameter, which is detectable as the user is executing the breathing exercise, the target breathing exercise may be adapted, e.g., according to the user's actual breathing capabilities, e.g., in order to increase the effectiveness of the breathing exercise, increase the user's comfort and compliance to the target breathing exercise, or at least increase the user's ability to comply with the target breathing exercise, during execution of the breathing exercise and/or prevent, or at least reduce, over-exertion, during execution of the breathing exercise. Moreover, monitoring breathing parameters in general may also provide a relatively reliable indication of changes to the user's physiological state. For instance, breathing information may provide an indication for exertion, fatigue and hypoxia during sports, general activity, daily life and sleep. Moreover, for instance, a person's breathing characteristics typically change when the person is under physical and/or psychological stress, e.g., the breathing may then be characterized by more frequent and shallower breaths. Thus, the effects of different breathing exercises on a person's physiological state may also be detected based on the breathing parameter, which is detectable as the user is executing the breathing exercise. Additionally, the target breathing exercise may also be adapted based on the physiological parameter which is detectable as the user is executing the breathing exercise, i.e., based on the effects of the breathing exercise on the user's physiological state. The breathing target breathing exercise may also be adapted based on subjective and/or objective user feedback and/or feedback by a medical professional, e.g., the difficulty and/or comfort and/or discomfort of a particular breathing exercise and/or an indication of a physiological, emotional and mental state before, during and/or after the breathing exercise as subjectively perceived by the user or objectively determined by the user, e.g., by means of detecting devices, for instance, by those included in the system described herein and/or by further external detecting devices.

Thus, the system described herein may improve the user's physiological state and/or mental state and/or emotional state effectively and/or efficiently and/or comfortably and/or free of over-exertion based on breathing exercises, which may be adapted and personalized based by the system.

For the purpose of adapting and/or personalizing the target breathing exercises to a specific user, the system may include a learning module which may be programmed to learn and adapt breathing exercise for each individual user. For this purpose, the system may include one or more processing means which the learning module cooperates with. The learning module may be configured to determine an optimal set of breathing exercises for each individual based on collected and/or accessed historical data related to the given user and/or to other users, e.g., users having similar physical attributes, such as age, size, weight and/or pre-existing conditions. The learning module may be configured to apply a mathematical close-form solution and/or an iterative algorithm to determine on or more personalized breathing exercises for the user. Additionally, or alternatively, the learning module may be configured to apply at least one of the following machine learning methods: decision tree, random forest, support vector machine, artificial neural network, linear regression, non-linear regression, deep learning, deep pattern recognition and statistical learning. When new data related to the user, e.g., subjective and/or objective user input/feedback and/or detected physiological parameters and/or further data, is available and accessible by system, the learning module may be configured to update the personalized breathing exercises by applying the optimization method anew considering the new data.

The system may be configured to estimate a corrective breathing pattern for daily life based on user inputs and the breathing parameter detected during real situations during a user's daily life, i.e., as a lifestyle device which may be worn during the day and/or night. Additionally, or alternatively, the system may provide general breathing guidance to the user, e.g., to be followed in one or more situations, so that user may apply such breathing guidance even when the system is not in use. Thus, the breathing guidance provided by the system may carry over into the user's daily life and daily routines without the system necessarily be operable when applying the breathing guidance. The system may still be configured to take the effects of the breathing guidance, which was followed when the system was not in use, on the user's physiological state and/or emotional state and/or mental state into account, e.g., in that the user may provide subjective and/or objective feedback to the system, e.g., via a user input interface, when the system is in use again.

The system may be configured to enable a pre- and/or post-check of the physiological parameters, in which the user is requested to breath naturally without breathing guidance. A person's breathing typically differs when the person is breathing consciously compared to when the person is breathing unconsciously. Thus, by enabling such a pre- and/or post-check of the physiological parameters when the user is breathing consciously, the target breathing metric and/or the target breathing exercise may be adapted based on physiological parameters and/or breathing parameters which are detectable during the pre- and/or post-check.

Preferably, the at least one physiological parameter includes at least one of the following: at least one cardiovascular parameter, at least one bioelectrical parameter, at least one parameter which is based on an analysis of at least one component in the user's sweat, and at least one temperature of the user's body, preferably a skin temperature and/or a core body temperature of the user. Such physiological parameters may provide a relatively reliable and quick responding indication of the user's physical and/or psychological state and/or general well-being.

The analysis of at least one component in the user's sweat may include determining the presence or absence of the component and/or an amount and/or a concentration of the component in the user's sweat. The analysis may be based on an electrodermal activity (EDA), a galvanic skin response (GSR) or an electrochemical biosensor measurement which the system is configured to perform.

Preferably, the at least one physiological parameter includes a cardiovascular parameter of at least one of the following types: a heart rate, a heart rate variability, a blood pressure and a blood oxygen level. The aforementioned cardiovascular parameters are well-established indicators for determining physical and/or psychological conditions, e.g., for an indication of a level or degree of an exercise and/or training and/or exertion, and may thus provide general information regarding the physical and/or psychological state of the user. The heart rate variability may be used as a biomarker for the state of the autonomous nervous system, both sympathetic and parasympathetic components, to give insights on both physical and mental well-being of the user. For instance, heart rate variability may be used as an objective assessment of psychological stress, preferably when monitored over time. Moreover, the reliability in determining heart rate variability by known technology, e.g., via an ECG/EKG, makes the heart rate variability a relatively reliable indicator for the physical and mental well-being of the user.

Preferably, the at least one target breathing index includes one or more of the following: a breathing frequency, a breath volume, a breath volume, a breath inhale volume, a breath exhale volume, an inspiratory time, an expiratory time, a breath pause time, a respiratory duty cycle, a total breath time, a breathing phase and a breathing pattern.

Preferably, the physiological monitoring device includes at least two electrodes configured to detect at least one electrocardiogram signal of the user. An electrocardiogram signal may be attained in a relatively reliable and comfortable manner, i.e., by applying the electrode in the vicinity of the user's heart, e.g., in a garment, preferably an upper body garment, which is wearable by the user or on the skin of the user's chest. Moreover, an electrocardiogram signal may allow a variety of parameters, e.g., cardiovascular parameters and/or breathing parameters and/or further physiological parameters, to be determined therefrom which may enhance the simplicity and efficiency of the physiological monitoring device. Exemplary electrodes which are integrated in a conductive fabric are described in EP 20 183 433.0, the content of which is herewith incorporated by reference in its entirety. EP 3 822 328 A1, the content of which is also incorporated herewith by reference in its entirety, describes an elastic seam tape, and a fabric including said seam tape, for enabling electronic capabilities in textiles, e.g., for connecting to sensors and/or electrodes, such as the electrodes described above. The electrodes and the elastic seam tape described in the above-identified European patent applications may be implemented in the system described herein.

Preferably, the physiological monitoring device and/or the breathing monitoring device is/are integrated in an item which is wearable by the user, preferably in a shirt and/or a belt. This may enhance the convenience and acceptability of the system described herein for everyday use, in particular since the physiological monitoring device and/or the breathing monitoring device may be relatively unobtrusive and/or less noticeable and/or better accepted when integrated in a wearable item.

Preferably, the breathing monitoring device includes a movement detection device for detecting movement of the chest and/or abdominal wall of the user as the user is breathing.

Preferably, the movement detection device includes a belt configured to be worn by the user and to provide respiratory inductance plethysmography. This may provide a relatively reliable and intuitive means for determining one or more breathing parameters.

Preferably, the physiological monitoring device is configured to continuously detect the physiological parameter of the user, and/or the breathing monitoring device is configured to continuously detect the breathing parameter of the user. A continuous detection, and thus monitoring, of the physiological parameter and/or breathing parameter may provide greater detail of the user's physical and/or psychological state in order to enhance the assessment and improvement of the user's physical and/or psychological state by means of the system described herein.

Preferably, the system is configured to provide instructions for executing the target breathing exercise. Preferably, the system is further configured to provide at least one of the following information to the user, preferably via at least one user interface: instructions for executing the target breathing exercise, the deviation between the target breathing index and the breathing parameter, which is detectable as the user is executing the breathing exercise, the detected physiological parameter, the detected breathing parameter and the change of the physiological parameter. The user interface may be integrated in the system as a component thereof. Alternatively, the user interface may be provided by a remote device, such as a smartwatch, smartphone, tablet, smart glasses, etc., which is connectable, and may communicate, with the system described herein.

Preferably, the user interface is configured to provide the information visually, haptically and/or audibly to the user. The information may be provided visually by displaying a text and/or pictures, such as a picture of a human who is executing the target breathing exercise, and/or animations.

Preferably, the system is configured to provide the information to the user substantially in real-time. This may provide a relatively quick response of the system in order to efficiently and effectively improve the user's physiological state.

Preferably, the system is configured to provide a visual animation of the target breathing exercise to the user, preferably via at least one user interface. This may enhance the quality of the instructions and/or the effectiveness of the instructions and/or the responsiveness of the user to the instructions.

Preferably, the system is configured to determine a breathing exercise score, i.e., an exercise compliance, based on the determined deviation between the target breathing index and the breathing parameter, which is detectable as the user is executing the breathing exercise. Optionally, the system may further be configured to adapt the target breathing exercise based on the determined deviation when the breathing exercise score is above or below a threshold, preferably a predetermined threshold. This may provide a relatively simple condition for determining whether the breathing exercise is being executed or has been executed in an optimal and/or effective manner. The breathing exercise score may be based on one or more breathing parameters which are detectable by the breathing monitoring device as the user is executing the breathing exercise.

The monitored actual breathing and the target breathing index may be represented by feature vectors to calculate the exercise compliance, i.e., a breathing exercise score. In order to measure how well the user is executing the breathing exercise, i.e. the exercise compliance (breathing exercise score), during a sequence of {t_0, t_1, . . . , t_n}, the system can compute the distance between the sequence of the actual breathing vector (denoted by ABV^{(t_0, t_n)}) of the user and the sequence of the target breathing vector (denoted by TBV^{(t_0, t_n)}) over the given sequence. The distance between these two sequences may be defined as the norm of the distance vector, wherein the element of the distance at index i vector is the norm of ABV^{t_i}−TBV^{t_i}, wherein i is in {0,1,2, . . . , n}. The norm can be any mathematical norm, for example L1_norm, L2_norm or Lp_norm for p>=1. The compliance parameter can be defined for example as 1/∥ABV^{t_i}−TBV^{t_i}∥ or −log (∥ABV^{t_i}−TBV^{t_i}∥). Alternatively, the compliance parameter may be defined for example as 1/∥ABV−TBV∥ or −log (∥ABV−TBV∥), wherein this norm may be the norm of the distance of two sequences.

Preferably, the target breathing exercise is adapted during the exercise, substantially in real-time such as breath by breath. This may increase the effectiveness of the breathing exercise relatively quickly such that the user must not execute the entire breathing exercise before receiving feedback on how well the breathing exercise is being executed. This may also aid in enhancing the user-friendliness and comfort of the system to the user and/or in preventing injury to the user when performing the breathing exercise, e.g., if the user is performing a breathing exercise which is beyond the user's physical capability, or is at or close to the user's physical capability.

Preferably, the system is configured to individually determine the target breathing exercise based on the target physiological index and/or the detected physiological parameter.

Preferably, the system is configured to determine an exercise effectiveness score based on a change of the detected physiological parameter and the breathing parameter which is detectable as the user is executing the breathing exercise.

The monitored actual breathing and the change of the physiological parameter(s) may be represented as feature vectors to calculate the exercise effectiveness (exercise effectiveness score). In order to measure how effective the actual breathing of the user is to influence the physiological parameter(s), i.e. the effectiveness of the breathing exercise, during a sequence of {t_0, t_1, . . . , t_n}, the system can compute the distance between the sequence of actual measured physiological feature vector (denoted by APV^{(t_0, t_n)}) and the sequence of the target physiological feature vector (denoted by TPV^{(t_0, t_n)}) over the given sequence. The distance between these two sequences may be defined as the norm of the distance vector, wherein the element of the distance at index i vector is the norm of APV^{t_i}-TPV^{t_i} where i is in {0,1,2, . . . , n}.

The norm can be any mathematical norm, for example L1_norm, L2_norm or Lp_norm for p>=1. The effectiveness parameter may be defined for example as 1/∥APV^{t_i}-TPV^{t_i}∥ or −log (∥APV^{t_i}−TPV^{t_i}∥). Alternatively, the effectiveness parameter may be defined for example as 1/∥APV−TPV∥ or −log (∥APV−TPV∥), wherein this norm may be the norm of the distance of two sequences.

Alternatively, the exercise effectiveness may be derived from the change in physiological parameters without taking the target physiological feature vector into account. The system may compute the distance between the sequence of actual measured physiological feature vector (denoted by APV^{(t_k, t_n)}) and the sequence of a time-shifted measured physiological feature vector (denoted by APV^{(t_0, t_m)}) during a sequence of {t_0, t_1, . . . , t_m, . . . , t_k, . . . , t_n}. The distance between these two sequences may be defined as the norm of the distance vector, wherein the element of the distance at index i vector is the norm of APV^{t_i}−APV^{t_e}, wherein is in {0,1,2, . . . , n} and e=i+k. The norm can be any mathematical norm, for example L1_norm, L2_norm or Lp_norm for p>=1. The effectiveness parameter can be defined for example as 1/∥APV^{t_i}−APV^{t_e}∥ or −log (∥APV^{t_i}−APV^{t_e}∥). Alternatively, the effectiveness parameter may be defined for example as 1/∥APV−TPV∥ or −log (∥APV−TPV∥), wherein this norm may be the norm of the distance of two sequences.

Preferably, the system is configured to determine, and optionally indicate to a user, a further target breathing exercise based on the exercise effectiveness score database, and optionally also on at least one physiological parameter of the user which is detectable by the physiological monitoring device.

Preferably, the system is configured to determine and/or adapt the target breathing exercise based on:
historical physiological data of the user which is accessible by the system;
and/or
user input provided by the user which is accessible by the system;
and/or
physical and/or physiological constraints of the user which is accessible by the system;
and/or
at least one physiological parameter of the user which is detectable by the physiological monitoring device as the user is executing the breathing exercise.

Preferably, the system is configured to adapt the target breathing exercise based on the user exercise compliance. If the breathing exercise is not conducted in the intended manner, the system may be configured to optionally give the user a first feedback that the exercise is not being performed or was not performed correctly and also optionally which breathing feature is not being performed or was not performed well enough. Furthermore, if the user repetitively remains below a compliance threshold, the systems may be configured to interpret this such as that the user cannot perform the exercise in a compliant manner. Therefore, the system may be configured to change the target breathing exercise towards the actual breathing of the user. For example, the system may be configured to adapt the target breathing vector by a linear or non-linear formula based on the actual breathing vector (ABV) and target breathing vector (TBV), such as:

$$New TBV = TBV + ( TBV - ABV ) * x ,$$

wherein x may be a predefined arbitrary coefficient.

Preferably, the system may be configured to learn the breathing capacity of the user as well as which breathing feature aspects the user cannot follow properly. Accordingly, the system may use this information to adapt the breathing exercises in the database and/or provide a new set of exercises tailored to the breathing capacity of the user.

Preferably, the system is configured to provide the user with one or more breathing exercise tests to determine at least one breathing capacity parameter including at least one of the following: a minimum breathing frequency, a maximum breathing frequency, a breathing volume, a breath inhale volume, a breath exhale volume, an inspiratory time, an expiratory time and a breath pause time.

Preferably, the system is configured to, based on the determined breathing capacity parameter, provide the user with one or more breathing exercises which are configured to improve at least one breathing capacity feature of the user.

Preferably, the system is configured to provide the user with one or more breathing exercise tests to determine at least one physiological capacity parameter including at least one of the following: a minimum heart rate, a maximum heart rate, a heart rate variability, a blood oxygen level, a CO2 tolerance, a CO2 level, and a stress level.

Preferably, the system is configured to, based on the determined physiological capacity parameter, provide the user with one or more exercises which are configured to improve at least one physiological capacity feature of the user.

Preferably, the system is configured to learn one or more physiological capacities and/or limits of the detected physiological parameters of the user. Accordingly, the system may be configured to use this information to adapt the breathing exercises in a database and/or the respective target physiological index in a database and/or provide one or more new exercises tailored to increase the physiological capacity of the physiological parameter of the user.

The selection of a target breathing exercise based on a target physiological target may be performed according to, but not limited to, the following. A database for each individual user contains data points for at least the following metrics: a target breathing vector (TBV), an actual breathing vector (ABV) which corresponds to the actual breathing activity performed by the user, a target physiological vector (TPV) and an actual physiological vector (APV). In case there is no individual user exercises stored yet, the database may be used with pre-defined common or reference values and/or exercises. Optionally, the database may contain subjective user feeling questionnaire answers labels before and/or after the breathing exercise which may be stored and used for the assessment. Based on machine learning, different models for different purposes can be learnt. For example, a machine learning model may be developed for each user and/or all users and/or a group of users for creating personalized breathing exercises based on the user breathing compliance, wherein the input of the model is a given target breathing index, e.g., a sequence of breathing feature vectors, and the output of the model is supposed to be the closet breathing exercise, e.g., a sequence of breathing feature vectors, to the given target which is compliant to the user. In order to apply this machine learning model, the stored TBV and the corresponding ABV data for each user and/or all users and/or a group of users in the database may be used to develop a machine learning model, respectively. model may be based on a classical machine learning technique and/or a deep learning method and/or linear regression and/or nonlinear regression. In case if in the database, we only have the breathing exercise parameter and not the target breathing exercise, one may use unsupervised learning to cluster different breathing pattern and to propose the optimal breathing exercise which is compliance for the user and is the closest one to the given target breathing index.

According to another example, in the database for the user, the recorded data for the target breathing exercises TBV1, TBV2, and TBV3, and the corresponding measured breathing parameters during these exercises are ABV1, ABV2, and ABV3, respectively, is provided. A simple linear machine learning model may be applied to propose the most compliant breathing exercise to a given target breathing vector, wherein TBV4=0.2*TBV1+05*TBV2+0.3*TBV3, then the model may propose ABV4=0.2*ABV1+05*ABV2+0.3*ABV3

A machine learning model may be developed for a user, wherein the input of the model is a given target physiological index (e.g. a sequence of physiological feature vector) and optionally at least one detected physiological parameter of the user at the starting point of the exercise and the output of the model is supposed to be an optimized/personalized breathing exercise (e.g. a sequence of breathing feature vectors) to achieve the given target physiological index. In order to develop this machine learning model, the stored APVs and the corresponding ABVs (and/or TBV) data points for the user and/or all users and/or a group of users in the database may be used to train a machine learning model, respectively. The machine learning model may be based on a classical machine learning technique and/or a deep learning method and/or linear regression and/or nonlinear regression.

For example, in the database for the user, the recorded data for the given breathing exercises ABV1, ABV2, and ABV3 and the achieved physiological indexes APV1, APV2, and APV3, respectively, is provided. A simple linear machine learning model may be applied, wherein, if the system wants to propose an exercise to achieve TPV4 where TPV4=0.2*TPV1+05*TPV2+0.3*TPV3, then the model may propose the breathing exercise corresponds to this breathing vector ABV4=0.2*ABV1+05*ABV2+0.3*ABV3

A machine learning model may be developed for a user, wherein the input of the model may be a given target subjective feeling of the user and the output of the model should be an optimal breathing exercise (e.g. a sequence of breathing feature vectors) to achieve the given target subjective feeling. In order to develop this machine learning model, the stored subjective feeling (SF) and the corresponding ABVs (and/or TBV) data points (where the subjective feeling has been collected after this breathing exercise for the user via a questionnaire) in the database to train a machine learning model, respectively, may be used. The machine learning model may be based on a classical machine learning technique and/or a deep learning method and/or linear regression and/or nonlinear regression.

For example, in the database for the user, if the user's subjective feeling after the given breathing exercises ABV1, ABV2, and ABV3 and denoted by SF1, SF2, and SF3, respectively, is provided/available, a simple linear machine learning model may be, if the system wants to propose an exercise to achieve SF4 where SF4=0.6*TPV1+0.4*TPV3, then the model may propose the breathing exercise corresponds to this breathing vector ABV4=0.6*ABV1+0.4*ABV3

The historical physiological data may include parameters which were detected by the system in the past and/or physiological data which was detected or determined in the past by other means outside of the system described herein. The historical physiological data may provide means for the system to learn and individually adapt to a user, e.g., like a biomarker which may give a general indication of, or at least a general change in, a physiological and/or emotional and/or mental state, which may be induced or achieved with the aid of one or more particular breathing exercises. For instance, it may be determined, based on the historical physiological data, that one or more particular breathing exercises are particularly effective in achieving a particular physiological and/or emotional and/or mental state. The historical physiological data may also provide an indication that one or more particular breathing exercises may be particularly effective in achieving a particular physiological and/or emotional and/or mental state based on one or more certain situations the user is currently, e.g., an initial physiological state and/or emotional state and/or mental state, and/or whether the particular physiological and/or emotional and/or mental state which the one or more particular breathing exercises may achieve or induce is desirable in the user's current situation. For instance, a certain physiological and/or emotional and/or mental state may not be desirable when the user is engaging in a leisure activity, as opposed to, for instance, when the user is conducting a job-related activity.

The system may include a user interface for providing the user input. The user input may include subjective and/or objective feedback by the user, e.g., the difficulty and/or comfort and/or discomfort of a particular breathing exercise and/or an indication of a physical, emotional and mental state before, during and/or after the exercise as subjectively perceived by the user or objectively determined by the user, e.g., by means of detecting devices, for instance, by those included in the system described herein and/or by further external detecting devices. Providing user input may increase the user-friendliness of the system, e.g., to maintain a balance between the challenge of the breathing exercise and user capabilities and/or user comfort. By maintaining such a balance, the breathing exercise is more likely to result in both an improved outcome and a more pleasant user experience. Thus, the system may provide gradual motivation to the user to increase the performance without over-exercising. Providing physical and/or physiological constraints of the user may also aid in preventing injuries to the user during execution of the breathing exercise.

Moreover, by including such user input/feedback, the breathing exercise(s) may be tailored and/or personalized to the user's needs and/or desires which may increase the effectiveness and/or efficiency of the breathing exercise and/or the user comfort and/or user compliance to the target breathing exercise during execution of the breathing exercise. By alternatively, or additionally, basing the determination and/or adaptation of the target breathing exercise on at least one physiological parameter of the user which is detectable by the physiological monitoring device as the user is executing the breathing exercise may allow to more precisely affect the user's physiological state in a positive manner by means of the breathing exercise. The user input and/or the physical and/or physiological constraints of the user may include physical attributes of the user, such as gender, age, general health condition, pre-existing health conditions, etc.

Preferably, the system is configured to determine and/or adapt the target breathing exercise based on at least one predetermined attribute of the user, preferably related to at least one of gender, age, height, weight, body mass index (BMI), preexisting illness or injury and ethnicity of the user.

Preferably, the system is configured to notify the user if the physiological parameter(s) detected by the physiological monitoring device and/or the breathing parameter(s) detected by the breathing monitoring device fulfill(s) at least one criterion, preferably a predetermined criterion, and determine, and optionally indicate to a user, a breathing exercise, based on the target breathing index and/or the target physiological index, respectively, wherein preferably the criterion includes the physiological parameter(s) and/or the breathing parameter(s) lying above or below a threshold, preferably a predetermined threshold. Thus, the system may determine, and optionally suggest, a breathing exercise once it has detected that the physiological state of the user has crossed a preset threshold. Thus, the system may monitor the user's physiological state in the background without actively determining, and optionally suggesting, a breathing exercise until the preset threshold has been crossed.

Preferably, the system is configured to provide breathing guidance to the user, preferably visually and/or acoustically, to change one or more characteristics of the user's breathing from an initial non-guided breathing to a target breathing, wherein the system is configured to adapt the breathing guidance gradually such that the user's breathing can be changed gradually from the non-guided breathing to the target breathing. The gradual adaptation of the breathing guidance may occur such that the breathing guidance is gradually adapted within the execution of the breathing exercise. For instance, the breathing guidance may call for the user, whose initial non-guided breathing includes 30 breaths per minute, to perform 24 breaths per minute in a first step. In a second step, which follows the first step, the breathing guidance may call for the user to perform 20 breaths per minute in a third step, and so forth. Thus, the breathing guidance may be adapted continuously or step-wise towards the target breathing. Additionally, or alternatively, the gradual adaptation of the breathing guidance may occur over longer periods of time, such as over days, weeks or months. In this case, the breathing guidance is gradually adapted from exercise to exercise, preferably in between at least some individual exercise. Overall, this may enable the user to gradually progress to the target breathing, which may make the breathing exercises more comfortable and/or may prevent over-exertion and/or injury to the user. This may increase the motivation, and thus the compliance, to perform the breathing exercises.

Preferably, the system is configured to provide guidance information to the user, preferably visually and/or acoustically, to guide the user through the target breathing exercise. The guidance information is preferably provided to the user at a speed which takes the user's physical breathing capabilities into account during execution of the breathing exercise by the user. This may provide a balance between a challenge of executing the breathing exercise and considering the user's physical capabilities. By maintaining the right balance, it is more likely to result in both an improved outcome and more pleasant user experience.

Preferably, the system may be configured to provide the user with a progressive and/or gradual breathing guidance to adapt the user's breathing habits towards a healthier breathing pattern during daily life and/or sleep with positive long-term effects with respect to a physiological state and/or a mental state and/or an emotional state of the user. For example, the system may be used to avoid and/or reduce chronic over-breathing and/or hyperventilation.

Preferably, the system may be configured to detect certain forms of breathing types such as diaphragmatic, deep, eupneic, costal, shallow breathing, hyperpnea, hyperventilation, hypoventilation or a breath pause/apnea. Optionally, the system my provide a notification to the user about the breathing type and optionally suggest a breathing exercise to be executed by the user, e.g., when the a specific breathing type is detected and/or the detected breathing parameter related to the breathing type are above or below a certain threshold, preferably a predetermined threshold.

Preferably, the system is configured to provide the breathing exercise to the user in an interactive game-like manner such that a virtual object in the game, preferably a movable object, which is visible and/or audible to the user, preferably via one or more screens and/or one or more microphones, is controllable by the user's breathing which is detectable by the breathing monitoring device. Preferably, a level of difficulty in the game and/or an aim in the game is based on one or more physiological parameters of the user. Providing such an interactive game-like aspect to the system may increase the user-friendliness and fun factor of the system which may increase the motivation of the user when using the system. Thus, the user's breathing may be used as a game controller during the game in order to control the virtual object, such as a movable car. For instance, a car may be maneuvered by the user's breathing, e.g., by complying to the target breathing exercise, e.g., in order to avoid objects. The objects may be arranged in such a manner to provide instructions/guidance for a target breathing.

The object mentioned at the beginning is also solved by a method for monitoring a physiological state of a user and providing at least one personalized breathing exercise to the user, as defined by the features of claim 31. Variations and further developments are defined by the features of the dependent claims. The features, configurations and advantages as described in relation to the system above apply to the method as well.

The method includes the following steps:
detecting at least one physiological parameter of the user by at least one physiological monitoring device;
detecting at least one breathing parameter of the user by at least one breathing monitoring device;
determining at least one target physiological index based at least on the detected physiological parameter;
determining a target breathing exercise to be executed by the user based at least on the target physiological index;
determining a target breathing index to be achieved by the user during execution of the breathing exercise;
comparing the target breathing index with the breathing parameter, which is detectable as the user is executing the breathing exercise, and determine a deviation therebetween; and
adapting the target breathing exercise based at least on the determined deviation.

Preferably, the at least one physiological parameter includes one or more of the following: a heart rate, a heart rate variability, a blood pressure and a blood oxygen level.

Preferably, the at least one target breathing index includes one or more of the following: a breathing frequency, a breath volume, a breath inhale volume, a breath exhale volume and a breathing phase.

Preferably, the physiological monitoring device continuously detects the physiological parameter of the user;

and/or the breathing monitoring device continuously detects the breathing parameter of the user.

Preferably, the method further includes the step of providing instructions for executing the target breathing exercise.

Preferably, the method further includes the step of providing at least one of the following information, preferably via at least one user interface: instructions for executing at least one target breathing exercise, the deviation between the target breathing index and the breathing parameter which is detectable as the user is executing the breathing exercise, the detected physiological parameter and the detected breathing parameter.

Preferably, the user interface provides the information visually, haptically and/or audibly to the user.

Preferably, the information is provided to the user substantially in real-time.

Preferably, a visual animation of the target breathing exercise is provided to the user, preferably via at least one user interface.

Preferably, a breathing exercise score is determined based at least on the determined deviation between the target breathing index and the breathing parameter, which is detectable as the user is executing the breathing exercise, and wherein the target breathing exercise is adapted based at least on the determined deviation when the breathing exercise score is above or below a threshold, preferably a predetermined threshold.

Preferably, the target breathing exercise is individually determined based on the target physiological index and/or the detected physiological parameter.

Preferably, the target breathing exercise is adapted based on:
historical physiological data of the user;
and/or
user input provided by the user;
and/or
physical and/or physiological constraints of the user
and/or
at least one physiological parameter of the user which is detectable by at least one physiological monitoring device as the user is executing the breathing exercise.

The object mentioned at the beginning is also solved by a virtual monitoring program for executing the method described herein as defined by the features of claim 43. Variations and further developments are defined by the features of the dependent claims. The features, configurations and advantages as described in relation to the system above apply to the method as well.

The monitoring program is configured to:
access at least one physiological parameter of the user which is detectable by at least one physiological monitoring device;
access at least one breathing parameter of the user which is detectable by at least one breathing monitoring device;
determine at least one target physiological index based on the detected physiological parameter;
determine a target breathing exercise to be executed by the user based on the target physiological index;
determine a target breathing index to be achieved by the user during execution of the breathing exercise;
compare the target breathing index with the breathing parameter, which is detectable as the user is executing the breathing exercise, and determine a deviation therebetween; and
adapt the target breathing exercise based on the determined deviation.

Preferably, the physiological parameter includes one or more of the following: a heart rate, a heart rate variability, a blood pressure and a blood oxygen level.

Preferably, the at least one target breathing index includes one or more of the following: a breathing frequency, a breath volume, a breath inhale volume, a breath exhale volume and a breathing phase.

Preferably, the monitoring program is configured to continuously access the physiological parameter and/or the breathing parameter of the user.

Preferably, the monitoring program is configured to provide instructions for executing the target breathing exercise.

Preferably, the monitoring program is configured to provide at least one of the following information to the user via a user interface: instructions for executing the target breathing exercise, the deviation between the target breathing index and the breathing parameter, which is detectable as the user is executing the breathing exercise, the detected physiological parameter and the detected breathing parameter.

Preferably, the monitoring program is configured to provide the information to the user substantially in real-time.

Preferably, the monitoring program is configured to provide a visual animation of the target breathing exercise to the user.

Preferably, the monitoring program is configured to determine a breathing exercise score based on the determined deviation between the target breathing index and the breathing parameter, which is detectable as the user is executing the breathing exercise, and wherein the monitoring program is configured to adapt the target breathing exercise based on the determined deviation when the breathing exercise score is above or below a threshold, preferably a predetermined threshold.

Preferably, the monitoring program is configured to individually determine the target breathing exercise based on the target physiological index and/or the detected physiological parameter.

Preferably, the monitoring program is configured to adapt the target breathing exercise based on:
historical physiological data of the user which is accessible by the monitoring program;
and/or
user input provided by the user which is accessible by the monitoring program;
and/or
physical and/or physiological constraints of the user which is accessible by the monitoring program;
and/or
at least one physiological parameter of the user which is detectable by the physiological monitoring device as the user is executing the breathing exercise.

Preferably, the monitoring program is installable and executable on a mobile device, preferably a device which is wearable by the user, preferably a smartwatch, smart glasses, a smartphone, a tablet and/or a pc.

Preferably, the monitoring program is configured to provide the breathing exercise to the user in an interactive game-like manner such that a virtual object in the game, preferably a movable object, which is visible and/or audible to the user, preferably via one or more screens and/or one or more microphones, is controllable by the user's breathing which is detectable by the breathing monitoring device. Preferably, a level of difficulty in the game and/or an aim in the game is based on one or more physiological parameters of the user.

The following list of aspects provides alternative and/or further features of the invention:
1. A system for monitoring a well-being of a user, preferably a physiological state and/or a mental state and/or an emotional state of the user, and providing at least one personalized breathing exercise to the user, the system including:
   at least one physiological monitoring device configured to detect at least one physiological parameter of the user; and
   at least one breathing monitoring device configured to detect at least one breathing parameter of the user;
wherein the system is configured to:
   determine at least one target physiological index, preferably based at least on the detected physiological parameter;
   determine a target breathing exercise to be executed by the user, preferably based at least on the target physiological index;
   determine a target breathing index to be achieved by the user during execution of the breathing exercise;
   optionally, compare the target breathing index with the breathing parameter, which is detectable as the user is executing the breathing exercise, and determine a deviation therebetween; and
   optionally, adapt the target breathing exercise, preferably based at least on user input and/or a physiological parameter which is detected as the user is executing the breathing exercise and/or a breathing parameter which is detected as the user is executing the breathing exercise and/or historical physiological and/or breathing data and/or the determined deviation.
2. The system according to aspect 1, wherein the at least one physiological parameter includes at least one of the following: at least one cardiovascular parameter, at least one bioelectrical parameter, at least one parameter which is based on an analysis of at least one component in the user's sweat, a skin conductance and/or a skin impedance of the user, a muscle activity, a general physical activity, a neuronal activity, a brain activity, a CO2 level and/or a partial pressure of CO2 in the blood and/or in the ventilated air of the user, a CO2 tolerance and/or a CO2 sensitivity of the user and at least one temperature of the user's body, preferably a skin temperature and/or a core body temperature.
3. The system according to aspect 1 or 2, wherein the system is configured to determine the target physiological index based at least on at least one physiological parameter and at least one breathing parameter.
4. The system according to any of the preceding aspects, wherein the at least one physiological parameter includes a cardiovascular parameter of at least one of the following types: a heart rate, a heart rate variability, a blood pressure, an arterial stiffness, an arterial elasticity, a pulse wave velocity and a blood oxygen level.
5. The system according to any of the preceding aspects, wherein the at least one target breathing index includes one or more of the following: a breathing frequency, a breath volume, a breath inhale volume, a breath exhale volume, an inspiratory time, an expiratory time, a breath pause time, a respiratory duty cycle, a total breath time, a breathing pattern and a breathing phase.
6. The system according to any of the preceding aspects, wherein the physiological monitoring device includes at least two electrodes configured to detect at least one electrocardiogram signal of the user.
7. The system according to any of the preceding aspects, wherein the physiological monitoring device and/or the breathing monitoring device is/are integrated in an item which is wearable by the user, preferably in a shirt.
8. The system according to any of the preceding aspects, wherein the breathing monitoring device includes a movement detection device for detecting movement of the chest and/or abdominal wall of the user as the user is breathing.
9. The system according to aspect 8, wherein the movement detection device includes a belt configured to be worn by the user and to provide respiratory inductance plethysmography, wherein preferably the belt is at least partially integrated in a shirt which is wearable by the user.
10. The system according to any of the preceding aspects, wherein the physiological monitoring device is configured to continuously detect the physiological parameter of the user;
and/or the breathing monitoring device is configured to continuously detect the breathing parameter of the user.
11. The system according to any of the preceding aspects, wherein the system is configured to provide instructions for executing the target breathing exercise.
12. The system according to any of the preceding aspects, further being configured to provide at least one of the following information to the user, preferably via at least one user interface: instructions for executing the target breathing exercise, the deviation between the target breathing index and the breathing parameter, which is detectable as the user is executing the breathing exercise, the detected physiological parameter, the detected breathing parameter and the change of the physiological parameter.
13. The system according to aspect 12, wherein the user interface is configured to provide the information visually, haptically and/or audibly to the user.
14. The system according to aspect 12 or 13, wherein the system is configured to provide the information to the user within 60 seconds, preferably within 30 seconds, more preferably within 20 seconds, more preferably within 10 seconds, more preferably within 5 seconds, more preferably within 1 second, more preferably within 500 ms, most preferably within 200 ms.
15. The system according to any of aspects 12 to 14, wherein the system is configured to provide the information to the user substantially in real-time.
16. The system according to any of the preceding aspects, wherein the system is configured to provide a visual animation of the target breathing exercise to the user, preferably via at least one user interface.
17. The system according to any of the preceding aspects, wherein the system is configured to determine a breathing exercise score based on the determined deviation between the target breathing index and the breathing parameter, which is detectable as the user is executing the breathing exercise, wherein the system is further configured to adapt the target breathing exercise based on the determined deviation when the breathing exercise score is above or below a threshold, preferably a predetermined threshold.
18. The system according to any of the preceding aspects, wherein the system is configured to individually determine the target breathing exercise based on the target physiological index and/or the detected physiological parameter.
19. The system according to any of the preceding aspects, wherein the target breathing exercise is adapted during execution of the breathing exercise, substantially in real-time, preferably in a breath by breath manner.
20. The system according to any of the preceding aspects, wherein the system is configured to determine an exercise effectiveness score based on a change of the detected physiological parameter and the breathing parameter which is detectable as the user is executing the breathing exercise.
21. The system according to aspect 20, wherein the system is configured to determine, and optionally indicate to a user, a further target breathing exercise based on the exercise effectiveness score database, and optionally also one at least one physiological parameter of the user which is detectable by the physiological monitoring device.
22. The system according to any of the preceding aspects, wherein the system is configured to determine and/or adapt the target breathing exercise based on:
   historical physiological data of the user which is accessible by the system;
   and/or
   user input provided by the user which is accessible by the system;
   and/or
   physical and/or physiological constraints of the user which is accessible by the system;
   and/or
   at least one physiological parameter of the user which is detectable by the physiological monitoring device as the user is executing the breathing exercise.
23. The system according to any of the preceding aspects, where the system is configured to determine and/or adapt the target breathing exercise based on at least one predetermined attribute of the user, preferably related to at least one of gender, age, height, weight, body mass index (BMI), preexisting illness or injury and ethnicity of the user.
24. The system according to any of the preceding aspects, wherein the system is configured to notify the user if the physiological parameter(s) detected by the physiological monitoring device and/or the breathing parameter(s) detected by the breathing monitoring device fulfill(s) at least one criterion, preferably a predetermined criterion, and determine, and optionally indicate to a user, a breathing exercise, based on the target breathing index and/or the target physiological index, respectively, wherein preferably the criterion includes the physiological parameter(s) and/or the breathing parameter(s) lying above or below a threshold, preferably a predetermined threshold.
25. The system according to any of the preceding aspects, wherein the system is configured to provide breathing guidance to the user, preferably visually and/or acoustically, to change one or more characteristics of the user's breathing from an initial non-guided breathing to a target breathing, wherein the system is configured to adapt the breathing guidance gradually such that the user's breathing can be changed gradually from the non-guided breathing to the target breathing.
26. The system according to any of the preceding aspects, wherein the system is configured to provide guidance information to the user, preferably visually and/or acoustically, to guide the user through the target breathing exercise, wherein the guidance information is provided to the user at a speed which takes the user's physical breathing capabilities into account during execution of the breathing exercise by the user.
27. The system according to any of the preceding aspects, wherein the system is configured to provide the breathing exercise to the user in an interactive game-like manner such that a virtual object in the game, preferably a movable object, which is visible and/or audible to the user, preferably via one or more screens and/or one or more microphones, is controllable by the user's breathing which is detectable by the breathing monitoring device, wherein preferably a level of difficulty in the game and/or an aim in the game is based on one or more physiological parameters of the user.
28. A method for monitoring a physiological state of a user, the method including the following steps:
   detecting at least one physiological parameter of the user by at least one physiological monitoring device;
   detecting at least one breathing parameter of the user by at least one breathing monitoring device;
   determining at least one target physiological index, preferably based at least on the detected physiological parameter;
   determining a target breathing exercise to be executed by the user, preferably based at least on the target physiological index;
   determining a target breathing index to be achieved by the user during execution of the breathing exercise;
   optionally, comparing the target breathing index with the breathing parameter, which is detectable as the user is executing the breathing exercise, and determine a deviation therebetween; and
   adapting the target breathing exercise, preferably based at least on user input and/or a physiological parameter which is detected as the user is executing the breathing exercise and/or a breathing parameter which is detected as the user is executing the breathing exercise and/or historical physiological and/or breathing data and/or the determined deviation.
29. The method according to aspect 28, wherein the at least one physiological parameter includes one or more of the following: a heart rate, a heart rate variability, a blood pressure, an arterial stiffness, an arterial elasticity, a pulse wave velocity and a blood oxygen level.
30. The method according to aspect 28 or 29, wherein the at least one target breathing index includes one or more of the following: a breathing frequency, a breath volume, a breath inhale volume, a breath exhale volume, an inspiratory time, an expiratory time, a breath pause time, a respiratory duty cycle, a total breath time, a breathing pattern and a breathing phase.
31. The method according to any of aspects 28 to 30, wherein the physiological monitoring device continuously detects the physiological parameter of the user;
   and/or
   the breathing monitoring device continuously detects the breathing parameter of the user.
32. The method according to any of aspects 28 to 31, further including the step of providing instructions for executing the target breathing exercise.
33. The method according to any of aspects 28 to 32, further including the step of providing at least one of the following information, preferably via at least one user interface: instructions for executing at least one target breathing exercise, the deviation between the target breathing index and the breathing parameter which is detectable as the user is executing the breathing exercise, the detected physiological parameter and the detected breathing parameter.
34. The method according to aspect 33, wherein the user interface provides the information visually, haptically and/or audibly to the user.
35. The method according to aspect 33 or 34, wherein the information is provided to the user substantially in real-time.
36. The method according to any of aspects 28 to 35, wherein a visual animation of the target breathing exercise is provided to the user, preferably via at least one user interface.
37. The method according to any of aspects 28 to 36, wherein a breathing exercise score is determined based at least on the determined deviation between the target breathing index and the breathing parameter, which is detectable as the user is executing the breathing exercise, and wherein the target breathing exercise is adapted based at least on the determined deviation when the breathing exercise score is above or below a threshold, preferably a predetermined threshold.
38. The method according to any of aspects 28 to 37, wherein the target breathing exercise is individually determined based on the target physiological index and/or the detected physiological parameter.
39. The method according to any of aspects 28 to 38, wherein the target breathing exercise is adapted based on:
   historical physiological data of the user;
   and/or
   user input provided by the user;
   and/or
   physical and/or physiological constraints of the user
   and/or
   at least one physiological parameter of the user which is detectable by at least one physiological monitoring device as the user is executing the breathing exercise.
40. A virtual monitoring program for executing the method according to any of aspects 28 to 39, the monitoring program being configured to:
   access at least one physiological parameter of the user which is detectable by at least one physiological monitoring device;
   access at least one breathing parameter of the user which is detectable by at least one breathing monitoring device;
   determine at least one target physiological index, preferably based on the detected physiological parameter;
   determine a target breathing exercise to be executed by the user, preferably based on the target physiological index;
   determine a target breathing index to be achieved by the user during execution of the breathing exercise;
   optionally, compare the target breathing index with the breathing parameter, which is detectable as the user is executing the breathing exercise, and determine a deviation therebetween; and
   adapt the target breathing exercise, preferably based at least on user input and/or a physiological parameter which is detected as the user is executing the breathing exercise and/or a breathing parameter which is detected as the user is executing the breathing exercise and/or historical physiological and/or breathing data and/or the determined deviation.
41. The monitoring program according to aspect 40, wherein the physiological parameter includes one or more of the following: a heart rate, a heart rate variability, a blood pressure, an arterial stiffness, an arterial elasticity, a pulse wave velocity and a blood oxygen level.
42. The monitoring program according to aspect 40 or 41, wherein the at least one target breathing index includes one or more of the following: a breathing frequency, a breath volume, a breath inhale volume, a breath exhale volume, an inspiratory time, an expiratory time, a breath pause time, a respiratory duty cycle, a total breath time, a breathing pattern and a breathing phase.
43. The monitoring program according to any of aspects 40 to 42, wherein the monitoring program is configured to continuously access the physiological parameter and/or the breathing parameter of the user.
44. The monitoring program according to any of aspects 40 to 43, wherein the monitoring program is configured to provide instructions for executing the target breathing exercise.
45. The monitoring program according to any of aspects 40 to 44, wherein the monitoring program is configured to provide at least one of the following information to the user via a user interface: instructions for executing the target breathing exercise, the deviation between the target breathing index and the breathing parameter, which is detectable as the user is executing the breathing exercise, the detected physiological parameter and the detected breathing parameter.
46. The monitoring program according to aspect 45, wherein the monitoring program is configured to provide the information to the user substantially in real-time.
47. The monitoring program according to any of aspects 40 to 46, wherein the monitoring program is configured to provide a visual animation of the target breathing exercise to the user.
48. The monitoring program according to any of aspects 40 to 47, wherein the monitoring program is configured to determine a breathing exercise score based on the determined deviation between the target breathing index and the breathing parameter, which is detectable as the user is executing the breathing exercise, and wherein the monitoring program is configured to adapt the target breathing exercise based on the determined deviation when the breathing exercise score is above or below a threshold, preferably a predetermined threshold.
49. The monitoring program according to any of aspects 40 to 48, wherein the monitoring program is configured to individually determine the target breathing exercise based on the target physiological index and/or the detected physiological parameter.
50. The monitoring program according to any of aspects 40 to 49, wherein the monitoring program is configured to adapt the target breathing exercise based on:
   historical physiological data of the user which is accessible by the monitoring program;
   and/or
   user input provided by the user which is accessible by the monitoring program;
   and/or
   physical and/or physiological constraints of the user which is accessible by the
   monitoring program;
   and/or
   at least one physiological parameter of the user which is detectable by the physiological monitoring device as the user is executing the breathing exercise.
51. The monitoring program according to any of aspects 40 to 50, wherein the monitoring program is installable and executable on a mobile device, preferably a device which is wearable by the user, preferably a smartwatch, smart glasses, a smartphone, a tablet and/or a pc.
52. The monitoring program according to any of aspects 40 to 51, wherein the monitoring program is configured to provide the breathing exercise to the user in an interactive game-like manner such that a virtual object in the game, preferably a movable object, which is visible and/or audible to the user, preferably via one or more screens and/or one or more microphones, is controllable by the user's breathing which is detectable by the breathing monitoring device, wherein preferably a level of difficulty in the game and/or an aim in the game is based on one or more physiological parameters of the user.

Preferred embodiments of the present invention are further elucidated below with reference to the figures. The described embodiments do not limit the present invention.

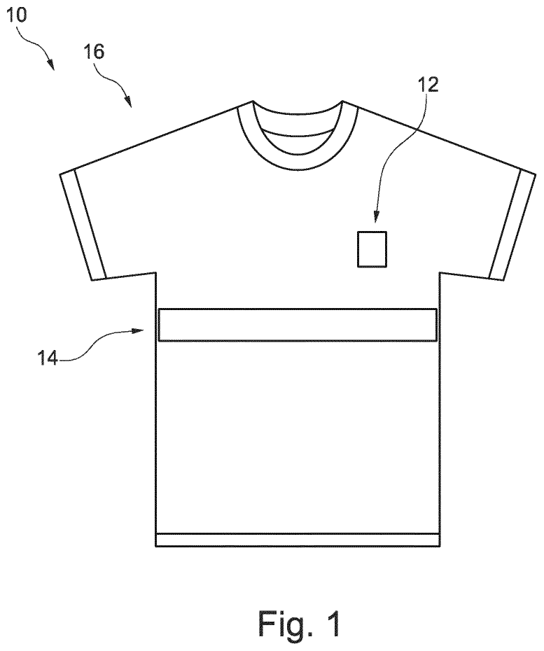

FIG. 1 shows a system according to an embodiment of the present invention;

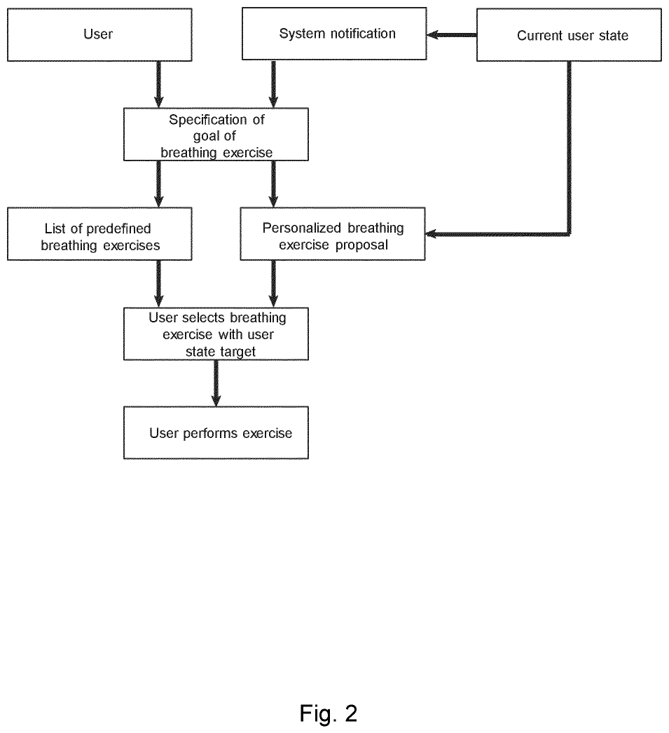

FIG. 2 shows a flow diagram related to a system according to the present invention, the flow diagram illustrating a possible breathing exercise selection process;

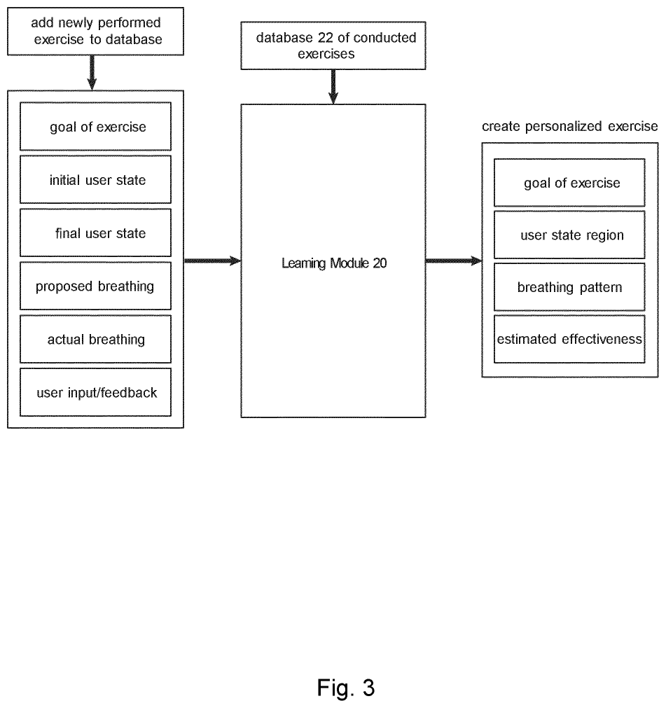

FIG. 3 shows a flow diagram related to a system according to the present invention, the flow diagram illustrating a possible learning module used for adaptation and personalization of breathing exercises;

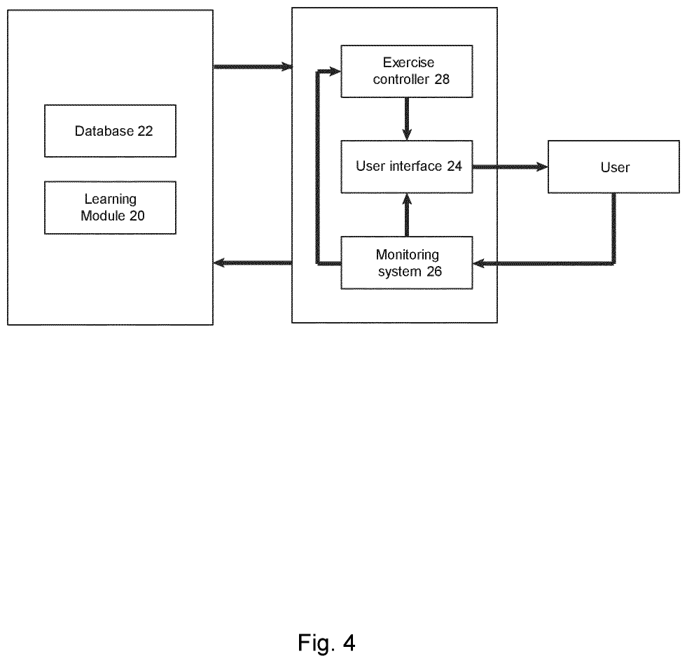

FIG. 4 shows a further flow diagram related to a system according to the present invention;

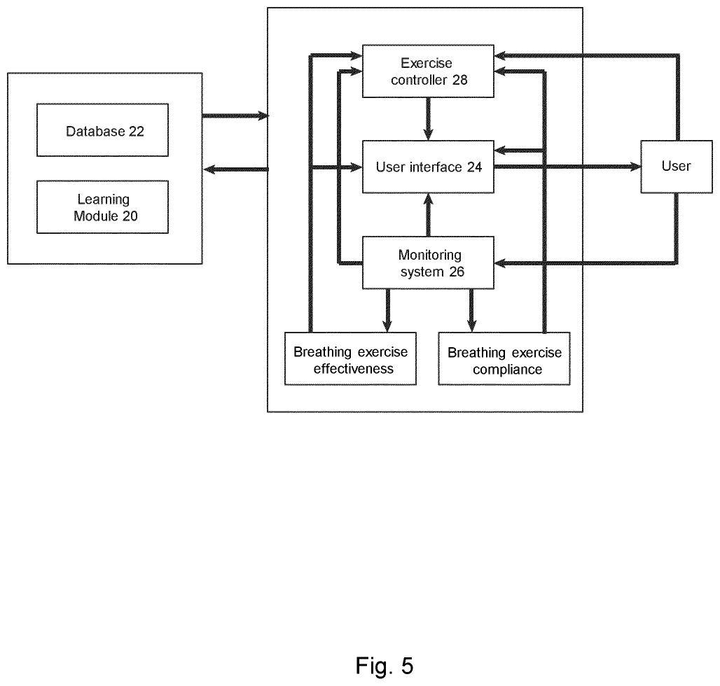

FIG. 5 shows a further flow diagram related to a system according to the present invention;

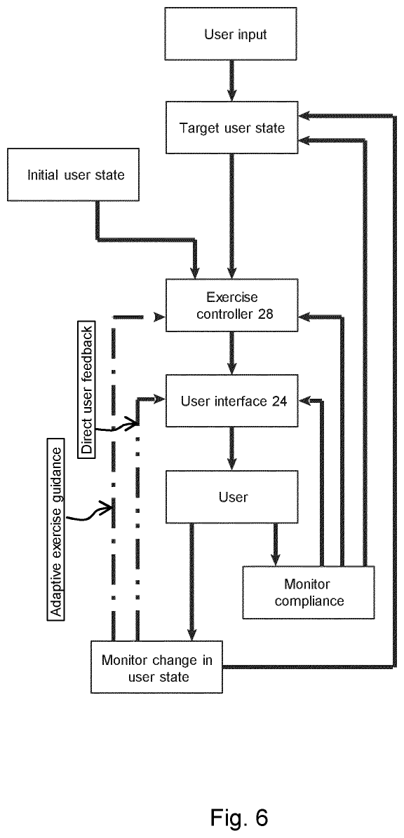

FIG. 6 shows a further flow diagram related to a system according to the present invention;

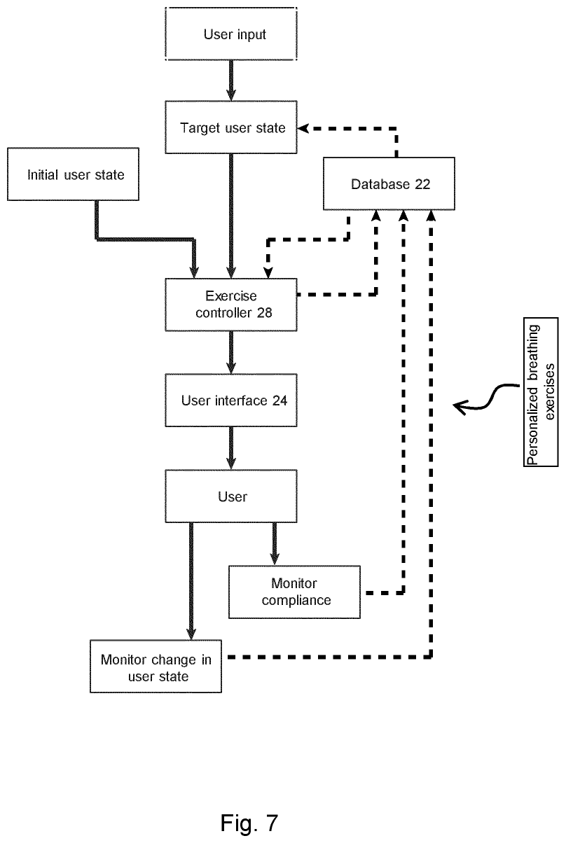

FIG. 7 shows a further flow diagram related to a system according to the present invention;

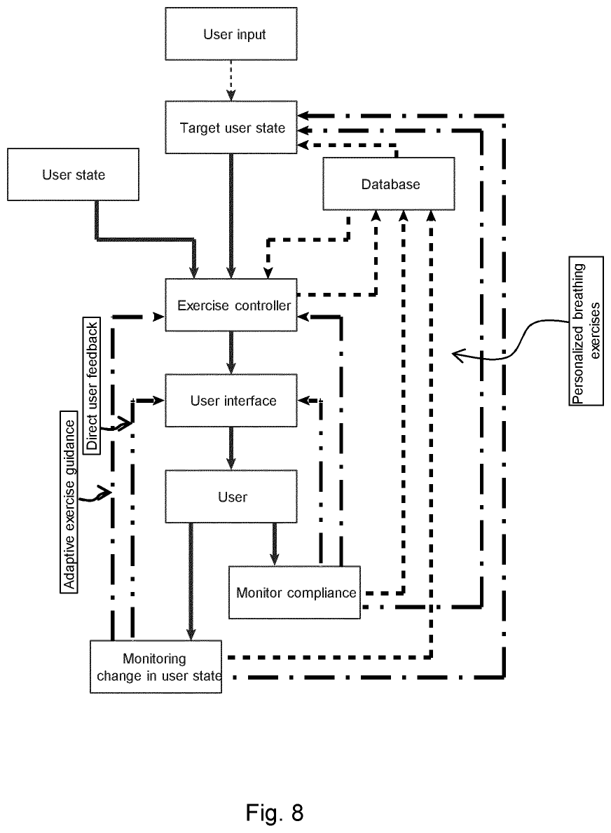

FIG. 8 shows a further flow diagram related to a system according to the present invention;

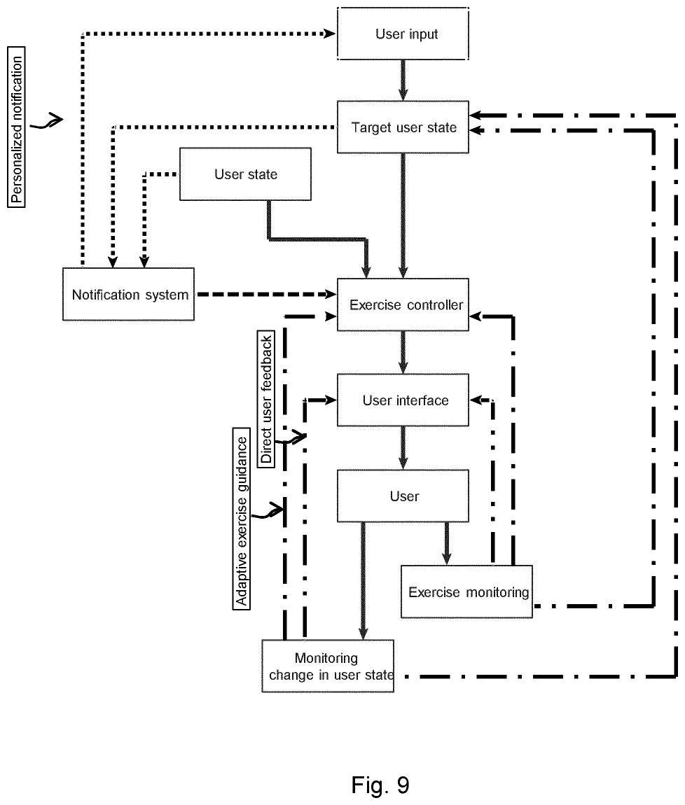

FIG. 9 shows a further flow diagram related to a system according to the present invention;

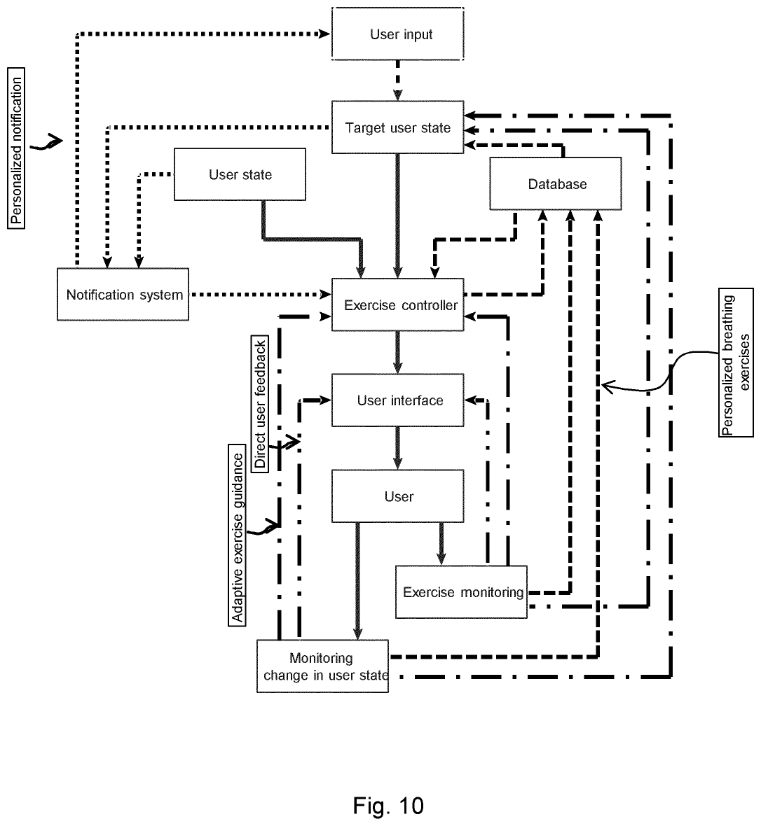

FIG. 10 shows a further flow diagram related to a system according to the present invention;

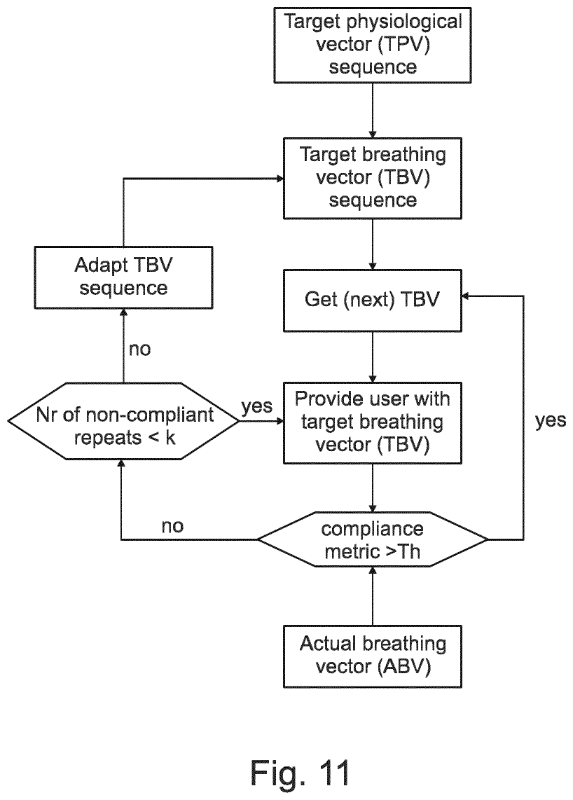

FIG. 11 shows a further flow diagram related to a system according to the present invention, in which a possible implementation of the system is shown;

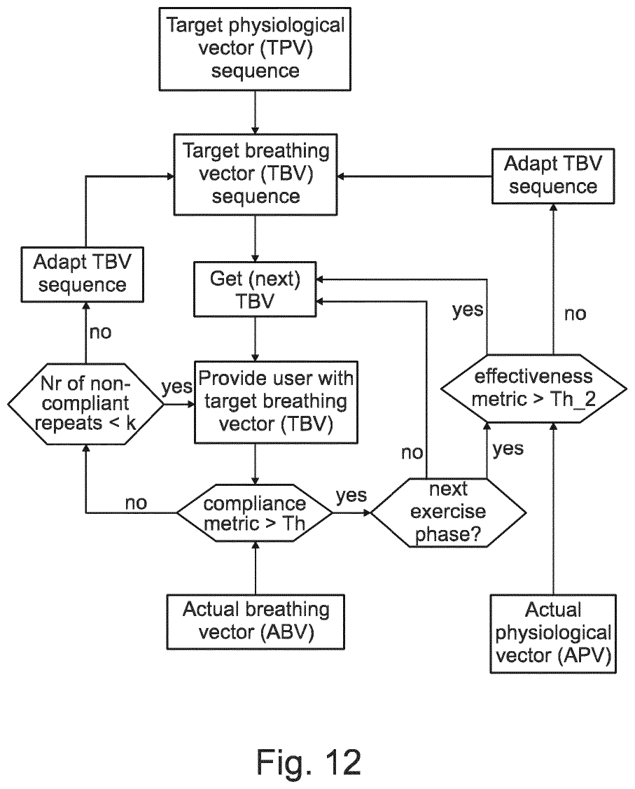

FIG. 12 shows a further flow diagram related to a system according to the present invention, in which a possible implementation of the system is shown.

FIG. 1 shows a system 10 according to an embodiment of the present invention. The system 10 is configured to monitor a physiological state and/or a mental state and/or an emotional state of a user and provide at least one personalized breathing exercise to the user, as described further below. The system 10 includes a physiological monitoring device 12 configured to detect at least one physiological parameter of the user and a breathing monitoring device 14 configured to detect at least one breathing parameter of the user. Alternatively, the physiological monitoring device 12 and the breathing monitoring device 14 may be configured as a single coherent unit. Preferably, the physiological monitoring device 12 and/or the breathing monitoring device 14, respectively, are configured as mobile devices such that the user can wear the physiological monitoring device 12 and/or the breathing monitoring device 14 and remain mobile meanwhile. As shown in the embodiment illustrated in FIG. 1, the system 10 is integrated in and/or attached to a garment 16, more specifically a shirt, configured to worn by the user. In particular, at least some of the components of the system 10 may be detachable from the garment 16, E.g., for easier replacement and/or servicing. Alternatively, only some of the components of the system 10 may be integrated in the garment 16, e.g., only the physiological monitoring device 12 or only breathing monitoring device 14. Alternatively, the system 10 including the physiological monitoring device 12 and the breathing monitoring device 14 may be integrated in or configured as any other wearable element(s), e.g., a watch, a belt, a hat, a backpack and/or a backpack. Further alternatively, the system 10 may not be integrated in a garment at all. For instance, the system may be configured as a stationary system, i.e., immobile relative to the user's movement.

The physiological monitoring device 12 may be configured to detect any physiological parameter, such as a cardiovascular parameter, e.g., a heart rate and/or a heart rate variability, e.g., by means of an electrocardiogramal, and/or a change in electrical conductance of the skin my occur in response to sweating by the user and/or a skin impedance, which may be detectable by means of electrochemical impedance spectroscopy and/or a general estimate of the general physical, mental and/or emotional state of the user, such as a level of stress, e.g., physical, psychological and/or oxidative stress, relaxation, fatigue, concentration, focus, surprise, happiness, depression, anxiety, excitement or other emotional state of the user. Alternatively, or additionally, the physiological parameter may be a muscle activity, e.g., which may be detectable by means of electromyography (EMG), and/or a general physical activity of the user, e.g. a jumping, running or walking movement, which may be detectable by means of an accelerometer, a gyroscope and/or a magnetometer and/or a neuronal activity and/or brain activity, e.g., which may be detectable by means of an Electroencephalogram (EEG) and/or a CO2 level and/or partial pressure of CO2, which may be detectable by means of a Capnography, included in the physiological monitoring device 12.

The system 10 is configured to determine at least one target physiological index based at least on the detected physiological parameter which is detectable by the physiological monitoring device 12. The target physiological index may be a certain physiological value of a specific physiological parameter, e.g., a specific value of a heart rate or a heart rate variability, and/or a minimum value and/or a maximum value. Additionally, or alternatively, the target physiological index may be a certain range of values, such as a range defined by a minimum value and a maximum value. The target physiological index may also be a combination of one or more values and/or one more ranged of values of one or more physiological parameters. Additionally, or alternatively, the target physiological index may be just a general direction of change of at least one physiological parameter of the user, e.g., a general reduction in the heart rate or an increase in heart rate variability of the user.

The system 10 is further configured to determine a target breathing exercise to be executed by the user based at least on the target physiological index. The target breathing exercise may include a breathing guidance provided to the user for controlling and/or altering one or more characteristics of the user's breathing, e.g., breathing frequency (respiratory rate) and/or inhale/exhale time.

Moreover, the system 10 is further configured to determine a target breathing index to be achieved by the user during execution of the breathing exercise. The system 10 is also configured to compare the target breathing index with the breathing parameter, which is detectable as the user is executing the breathing exercise, and determine a deviation therebetween. The system 10 is further configured to adapt the target breathing exercise based at least on the determined deviation.

The system 10 described herein may improve the user's physiological state and/or mental state and/or emotional state effectively and/or efficiently and/or comfortably and/or free of over-exertion based on breathing exercises which may be adapted and personalized by the system to provide personalized breathing exercises to the user.

FIG. 2 shows a flow diagram related to the system 10 shown in FIG. 2. In particular, the flow diagram of FIG. 2 illustrates a possible breathing exercise selection process. The system 10 may determine a current user state, e.g., a physiological state and/or a mental state and/or an emotional state, based on one or more physiological parameters and/or one or more breathing parameters detected by the physiological monitoring device 12 and the breathing monitoring device 14, respectively, and/or on user input. The system 10 may then be notified that a breathing exercise should be executed by the user, e.g., when the detected physiological parameter and/or the detected breathing parameter are above or below a certain threshold, preferably a predetermined threshold. Alternatively, the user may indicate a desire to execute a breathing exercise, e.g., via a user input interface.

The user may specify the goal of the breathing exercise, e.g., via a user input interface. Alternatively, the system 10 may be configured to determine goal of the breathing exercise, e.g., based on the detected physiological parameter and/or the detected breathing parameter. For instance, the goal may be determined by the system 10 as being a general improvement of the detected physiological state and/or a mental state and/or an emotional state.

The user may select, e.g., via a user input interface, a breathing exercise from a list of predefined breathing exercises and/or the system may propose a personalized breathing exercise, e.g., based on the detected physiological parameter and/or the detected breathing parameter. The user may select the breathing exercise from the list of predefined breathing exercises or the personalized breathing exercise. The user may then perform the selected breathing exercise.

Alternatively, the system 10 may be configured to directly propose a personalized breathing exercise based on the detected physiological parameter and/or the detected breathing parameter.

FIG. 3 shows a flow diagram related to the system shown in FIG. 1. The system 10 may include a learning module 20, as shown in FIG. 3. The learning module 20 is configured to adapt and personalize breathing exercises. The learning module may be configured to access a database 22 of conducted exercise including historical data, such as physiological and/or breathing parameters which were detected before, during and/or after said conducted exercises. Once a breathing exercise has been performed, the breathing exercise may be added to the database. Information of the breathing exercise, such as a goal of the breathing exercise, an initial user state, e.g., a physiological state and/or a mental state and/or an emotional state before starting the breathing exercise, a final user state, e.g., a physiological state and/or a mental state and/or an emotional state before finishing the breathing exercise, a proposed breathing, i.e., according to a target breathing exercise as proposed by the system 10, an actual breathing and user input/feedback, may be included in the database. Based on the received information, the learning module 20 may adapt and personalize breathing exercises based on, e.g., a goal of the breathing exercise, a user state region, e.g., a range of user states which may be categorized to one common user state region, a breathing pattern and an estimated effectiveness of the breathing exercise. The user state region may include a lookup table in which the user state region may be determined.

FIG. 4 shows a further flow diagram related to the system shown in FIG. 1.

According to the flow diagram of FIG. 4, the system 10 includes a user interface 24, a monitoring system 26, which includes the physiological monitoring device 12 and the breathing monitoring device 14, and an exercise controller 28. The monitoring system 26, e.g., the physiological monitoring device 12 and the breathing monitoring device 14, is configured to detect/measure various data of the user, e.g., various parameters of the user, such as physiological parameters and/or breathing parameters and/or any other parameters related to the well-being of the user. The system 10 is configured to provide the data, e.g., the physiological parameters and/or breathing parameters and/or any other parameters related to the well-being of the user, to the learning module 20 and the database 22, e.g., such that the system may generate personalized breathing exercises based on said data. The user interface 24 may provide information, such as breathing guidance related to a target breathing exercise, to the user, e.g., in visual, preferably animated, acoustic and/or haptic form. The user interface 24 may include a screen and/or audio means, such as one or more speakers and/or an audio signal outlet to provide an audio signal to an external device. The exercise controller 28 is configured to determine, and optionally suggest to the user, one or more breathing exercises based on, e.g., historical data in the data base 22, physiological parameters and/or breathing parameters and/or any other parameters related to the well-being of the user which are or were detected by the physiological monitoring device 12 and the breathing monitoring device 14. For this purpose, the exercise is communicatively connected to the monitoring system 26, the learning module 20 and the database 22.

FIG. 5 shows a further flow diagram based on the flow diagram shown in FIG. 4. In addition to the flow diagram shown in FIG. 4, the flow diagram of FIG. 5 shows that the system 10 may also provide information related to breathing exercise compliance and breathing exercise effectiveness. The breathing exercise compliance and/or breathing exercise effectiveness may be determined by comparing a target breathing index with a breathing parameter, which is detectable as the user is executing the breathing exercise, and determining a deviation therebetween. Thus, if the deviation is relatively large, it may generally be assumed that the user is not complying with the breathing guidance, i.e., the user is not correctly performing a target breathing exercise, and/or that the target breathing exercise is not as effective in improving the physiological state and/or mental state and/or emotional state as desired or required. For instance, a certain deviation threshold may be predetermined to indicate insufficient compliance and/or effectiveness, if the determined deviation is above the predetermined threshold. Moreover, the information related to the breathing exercise compliance and the breathing exercise effectiveness may also be provided to the user interface 24 to provide the information to the user, as shown in FIG. 5. In addition, the user may interact directly with the exercise controller 28, as also shown in FIG. 5, e.g., by providing user input to the exercise controller to directly or indirectly determine, at least partially, the target breathing exercise to be performed.

FIG. 6 shows a further flow diagram related to the system 10 described herein. Based on an initial user state, e.g., a physiological state and/or a mental state and/or an emotional state before starting a breathing exercise, which may be determined based on the physiological parameters and/or the breathing parameters detected by the physiological monitoring device 12 and the breathing monitoring device 14 and/or user input, the exercise controller 28 may determine and propose a target breathing exercise to the user, e.g., by providing breathing guidance to the user via the user interface 24. Compliance of the user with respect to the target breathing exercise may be monitored (“monitor compliance” in FIG. 6) and the user state, more specifically a change in the user state, may also be monitored (“monitor change in user state” in FIG. 6), as described above. Based thereon, the compliance of the user and the effectiveness of the performed breathing exercise may be determined by the system 10. The user may provide direct user feedback to the system with respect to the monitoring of the user state, more specifically a change in the user state. Hence, the monitoring of a change in the user state may also be based on direct user feedback in addition to the data detected by the monitoring device 12 and the breathing monitoring device 14. The information related to the monitored change in the user state is provided to the exercise controller 28, based on which adaptive exercise guidance may be generated and provided to the user, as the user is still performing the breathing exercise and/or for one or more subsequent breathing exercises. Information regarding a target user state, e.g., a physiological state and/or a mental state and/or an emotional state of the user which is to be achieved by performing the target breathing exercise, is also provided by the system. The user may also influence the target user state by providing user input related to the target user state.

FIG. 7 shows a further flow diagram based on the flow diagram shown in FIG. 6. In addition to the flow diagram shown in FIG. 6, the flow diagram of FIG. 7 further includes the database 22, as described above with respect to FIGS. 3 to 5. As indicated by the dashed lines in FIG. 7, the system 10 may be configured such that the compliance monitoring, e.g., by means of detection by the breathing monitoring device 14, the monitoring of a change in the user state, e.g., by means of detection by the physiological monitoring device 12, the database 22 and the exercise controller interact with each other to generate personalized breathing exercise, as described herein.

FIG. 8 shows a further flow diagram based on the flow diagram shown in FIG. 7. In addition, the system 10 is configured to provide adaptive exercise guidance, which is indicated by dash-dot-lines in FIG. 8, and direct user feedback, which is indicated by dash-dot-dot-lines in FIG. 8, as described above with respect to FIG. 6.

FIG. 9 shows a further flow diagram based on the flow diagram shown in FIG. 6. In addition, the system 10 includes a notification system configured to provide a personalized notification to the user, which is indicated by dashed lines in FIG. 9. The notification system may be configured to continuously monitor the user state and give the user a notification, e.g., by visual, haptic and/or audio feedback, if at least one detected physiological parameter, such as a heart rate, a heart rate variability, a stress level, a blood pressure, a blood oxygen level, etc., and/or at least one detected breathing parameter, such as a a breathing rate, a breath volume, a breath pause time is above or below a certain threshold, preferably a predetermined threshold and/or if the system detects a breathing type such as diaphragmatic, deep, eupneic, costal, shallow breathing, hyperpnea, hyperventilation, hypoventilation or an elongated breath pause/apnea and/or any combination between breathing type and/or physiological parameter and/or breathing parameter. Optionally, the notification system may be configured to request the user to give an input such as the user's current subjective feeling and/or if the user would like to do a proposed breathing exercise. Optionally, the system may change the target breathing exercise in case the user is currently conducting a breathing exercise. Alternatively, the notification system may also be configured to provide the user with notifications during an exercise based on the actual physiological parameter and/or the actual breathing parameter and/or the target breathing parameter and/or the target physiological parameter and/or the exercise compliance and/or the exercise effectiveness based on predetermined thresholds.

FIG. 10 shows a further flow diagram which essentially represents a combination of the configuration shown in FIGS. 7 and 9.

FIG. 11 shows a flow diagram of a possible implementation of the system described herein, in particular how the system may adapt the breathing exercise based on a user breathing compliance or non-compliance, respectively. The system may be configured to define a sequence of target breathing vectors (TBV) based on a sequence of target physiological vectors (TPV). The user is provided with the information of the current TBV while the actual breathing vector (ABV) of the user is monitored. The system is configured to check if the breathing compliance is above a compliance threshold (Th). If yes, the system may continue with providing the user with the next TBV. If no, the system continues with the same TBV unless the compliance threshold (Th) was not reached for a total of k repeats. Once the compliance threshold was not reached for k times, the system adapts the TBV sequence to adapt the exercise closer to the ABV.

FIG. 12 shows a flow diagram of a further possible implementation of the system described herein, in particular how the system may adapt the breathing exercise based on the user breathing compliance and the exercise effectiveness. The system may be configured to define a sequence of target breathing vectors (TBV) based on a sequence of target physiological vectors (TPV). The user is provided with the information of the current TBV while the actual breathing vector (ABV) of the user is monitored. The breathing compliance may be used to adapt the TBV sequence as described in FIG. 11. In addition, as long as the exercise compliance is higher than the compliance threshold (Th), the system may calculate the exercise effectiveness each time the system enters a new exercise phase. This exercise phase information may be provided as additional information in the TPV sequence. Once a new phase starts, the exercise effectiveness is calculated and compared to an effectiveness score threshold (Th_2). If the exercise effectiveness score is above Th_2, the exercise continues with the next TBV. If the exercise was not effective, the TBV sequence may be adapted to increase the potential effectiveness of the exercise.

Various breathing exercise examples are described below to illustrate possible implementations of the system.

### Breathing Exercise Example 1

The breathing exercise may be conducted in a breath by breath manner. The exercise may be divided in different stages. In the first stage, a heart rate variability (HRV) is measured as the physiological parameter during normal breathing of the user for a time duration of 1-5 minutes. A derived HRV value is used to set the physiological target index, which may be an HRV value that is substantially higher. The selection may be conducted by using the user database, whereas the system checks for exercises with an initial HRV in the similar region as the current HRV (for example current HRV+/−10 ms) of the user and selects a target HRV value that corresponds to the highest final HRV in the corresponding exercises. Optionally, the HRV target may be increased by a predefined value such as 10 ms. The system is configured to then determine the corresponding target breathing exercise based on the target HRV value. Optionally, the system may use the actual breathing vector in addition to the target HRV to determine a target breathing exercise. The selection process may also be a database look-up where an exercise with the highest effectiveness score is selected for the current HRV and/or current breathing frequency. In the second stage, the user follows the target breathing exercise. The system may be configured to check if the actual breathing parameter, such as frequency, is compliant with the target breathing frequency and adjust the target breathing frequency substantially in real-time if the user does not or cannot follow the exercise.

Optionally, the system is configured to adjust the target breathing exercise based on changes in the detected physiological parameter, e.g., the HRV. Optionally, in a third stage, the HRV is measured as the physiological parameter during normal breathing of the user for a time duration of 1-5 minutes.

### Breathing Exercise Example 2

The breathing exercise is conducted in a breath by breath manner. The exercise may be divided in different stages. First, the goal of the exercise is selected, such as stress level reduction in case of a high current stress level of the user. In a first phase, the stress level is measured during normal breathing of the user for a time duration of 1-5 minutes. Optionally, the user may provide at least one user input related to the user's current subjective feeling. The system may be configured to determine the target stress level based on the stress level measured before and/or during the first phase of the breathing exercise and, optionally, additionally based on the breathing parameter measured before and/or during the first phase of the breathing exercise. The target breathing exercise may be determined based on the target stress level, and optionally additionally based on the breathing parameter measured before and/or during the first phase of the breathing exercise. The selection process may be a database look-up where an exercise with the highest effectiveness score is selected for the current stress level additionally taking into account the user breathing compliance by using a machine learning model and the user database. In a second stage, if the breathing rate of the user is more than 5 breaths per minute higher than a target breathing rate, the system is configured to perform a breath by breath breathing exercise adaption. The target breathing rate sequence may contain target breathing rates that decrease by 2 seconds after each breathing cycle performed correctly, i.e., in a compliant manner, until the target breathing rate is reached. In a third stage, the user follows the target breathing exercise. If the breathing is not compliant, the target breathing vector sequence may be adjusted. As long as the breathing is compliant, the system may monitor the stress level of the user and the change of stress level of the user. As long as the stress level changes towards the target stress level, the system continues with the next target breathing vector of the sequence. If the stress level moves away from the target stress level, the target breathing vector can be selected among previous breathing vector with a positive change or based on a database comparison to select an alternative target breathing vector sequence. Optionally, in a fourth phase, the stress level is again measured during normal breathing of the user for a time duration of 1-5 minutes. Optionally, the user may provide at least one user input related to the user's current subjective feeling. Finally, at least the target breathing vector sequence, the actual breathing vector sequence, the target physiological vector and the actual physiological vector are stored in the database. Optionally, the learning module may create a new personalized breathing exercise based on the various vectors, the estimated exercise effectiveness and the estimated exercise compliance.

### Breathing Exercise Example 3

The breathing exercise may be conducted in a breath by breath manner. The exercise may be divided in different stages. In a first stage, the user optionally conducts a control breath pause check. The user breaths normally (non-guided) for 1-5 minutes. After a normal exhale, the user is requested by the system to hold the user's breath until the user feels the urge to breath. The system detects the inhale and determines the user breath pause time. The detected physiological parameter may be a CO2 tolerance estimated from the breath pause time. Optionally, the user can give the system a user input with respect to this time. The physiological parameter is then used to determine the target physiological index, for example a target CO2 tolerance which is substantially higher. The target CO2 tolerance is used to select a target breathing exercise, such as slow and light/shallow breathing. The breathing pattern is slowly reduced in breathing frequency and breathing volume as long as the user can comply with it while an additional physiological parameter such as heart rate is used to avoid hypoventilation. In this sense, the breathing compliance and the additional physiological parameter are used to keep the exercise in balance. This information may also be stored to create a personalized breathing exercise for the next breathing session.

Alternatively, the same exercise may be performed with a physiological parameter such as a heart rate.

1. A system for monitoring at least one of a physiological state, a mental state, or an emotional state of a user and providing at least one personalized breathing exercise to the user, the system including:
at least one physiological monitoring device configured to detect at least one physiological parameter of the user; and
at least one breathing monitoring device configured to detect at least one breathing parameter of the user;
wherein the system is configured to:
determine at least one target physiological index based at least on the detected physiological parameter;
determine a target breathing exercise to be executed by the user based at least on the target physiological index;
determine a target breathing index to be achieved by the user during execution of the breathing exercise;
compare the target breathing index with the breathing parameter, which is detectable as the user is executing the breathing exercise, and determine a deviation therebetween; and
adapt the target breathing exercise based at least on the determined deviation.

2. The system according to claim 1, wherein the at least one physiological parameter includes at least one of the following: at least one cardiovascular parameter, at least one bioelectrical parameter, at least one parameter which is based on an analysis of at least one component in the user's sweat, a skin conductance of the user, a skin impedance of the user, a muscle activity, a general physical activity, a neuronal activity, a brain activity, a CO2 level of CO2 in the blood of the user, a partial pressure of CO2 in the blood of the user, a partial pressure of CO2 in the ventilated air of the user, a CO2 tolerance of the user, a CO2 sensitivity of the user, at least one temperature of the user's body, a heart rate, a heart rate variability, a blood pressure, an arterial stiffness, an arterial elasticity, a pulse wave velocity, or a blood oxygen level.

3. The system according to claim 1, wherein the system is configured to determine the target physiological index based at least on at least one physiological parameter and at least one breathing parameter.

4. (canceled)

5. The system according to claim 1, wherein:
the at least one target breathing index includes one or more of the following: a breathing frequency, a breath volume, a breath inhale volume, a breath exhale volume, an inspiratory time, an expiratory time, a breath pause time, a respiratory duty cycle, a total breath time, a breathing phase or a breathing pattern.

6. (canceled)

7. The system according to claim 1, wherein at least one of the physiological monitoring device or the breathing monitoring device is integrated in an item which is wearable by the user.

8. The system according to claim 1, wherein the breathing monitoring device includes a movement detection device for detecting movement of at least one of the chest or abdominal wall of the user as the user is breathing.

9. The system according to claim 8, wherein the movement detection device includes a belt configured to be worn by the user and to provide respiratory inductance plethysmography.

10.-11. (canceled)

12. The system according to claim 1, further being configured to provide at least one of the following information to the user via at least one user interface: instructions for executing the target breathing exercise, the deviation between the target breathing index and the breathing parameter, which is detectable as the user is executing the breathing exercise, the detected physiological parameter, the detected breathing parameter, or the change of the physiological parameter.

13. (canceled)

14. The system according to claim 12, wherein the system is configured to provide the information to the user.

15. The system according to claim 12, wherein the system is configured to provide the information to the user substantially in real-time.

16. The system according to claim 1, wherein the system is configured to:
provide a visual animation of the target breathing exercise to the user via at least one user interface.

17. The system according to claim 1, wherein the system is configured to determine a breathing exercise score based on the determined deviation between the target breathing index and the breathing parameter, which is detectable as the user is executing the breathing exercise, wherein the system is further configured to adapt the target breathing exercise based on the determined deviation when the breathing exercise score is above or below a predetermined threshold.

18. (canceled)

19. The system according to claim 1, wherein the target breathing exercise is adapted during execution of the breathing exercise, substantially in real-time or in a breath-by-breath manner.

20. The system according to claim 1, wherein the system is configured to determine an exercise effectiveness score based on a change of the detected physiological parameter and the breathing parameter which is detectable as the user is executing the breathing exercise.

21. (canceled)

22. The system according to claim 1, wherein the system is configured to determine and adapt the target breathing exercise based on at least one of:
historical physiological data of the user which is accessible by the system;
user input provided by the user which is accessible by the system;
physical constraints of the user which is accessible by the system;
at least one physiological parameter of the user which is detectable by the physiological monitoring device as the user is executing the breathing exercise; or
at least one predetermined attribute of the user related to at least one of gender, age, height, weight, body mass index (BMI), preexisting illness or injury, or ethnicity of the user.

23.-24. (canceled)

25. The system according to claim 1, wherein the system is configured:
to provide breathing guidance to the user to change one or more characteristics of the user's breathing from an initial non-guided breathing to a target breathing, wherein the system is configured to adapt the breathing guidance gradually such that the user's breathing can be changed gradually from the non-guided breathing to the target breathing.

26. (canceled)

27. The system according to claim 1, wherein the system is configured to provide the user with one or more breathing exercise tests to determine:
at least one breathing capacity parameter including at least one or more of the following: a minimum breathing frequency, a maximum breathing frequency, a breathing volume, a breath inhale volume, a breath exhale volume, an inspiratory time, an expiratory time and a breath pause time.

28. The system according to claim 1, based on the determined breathing capacity parameter, the system is configured to provide the user with one or more breathing exercises which are configured to improve at least one of at least one breathing capacity feature of the user or at least one physiological capacity feature of the user.

29.-31. (canceled)

32. A method for monitoring a well-being of a user and providing at least one personalized breathing exercise to the user, the method including the following steps:
detecting at least one physiological parameter of the user by at least one physiological monitoring device;
detecting at least one breathing parameter of the user by at least one breathing monitoring device;
determining at least one target physiological index based at least on the detected physiological parameter;
determining a target breathing exercise to be executed by the user based at least on the target physiological index;
determining a target breathing index to be achieved by the user during execution of the breathing exercise;
comparing the target breathing index with the breathing parameter, which is detectable as the user is executing the breathing exercise, and determine a deviation therebetween; and
adapting the target breathing exercise based at least on the determined deviation.

33.-43. (canceled)

44. A virtual monitoring program for executing the method according to claim 32, the monitoring program being configured to:
access at least one physiological parameter of the user which is detectable by at least one physiological monitoring device;
access at least one breathing parameter of the user which is detectable by at least one breathing monitoring device;
determine at least one target physiological index based at least on the detected physiological parameter;
determine a target breathing exercise to be executed by the user based at least on the target physiological index;
determine a target breathing index to be achieved by the user during execution of the breathing exercise;
compare the target breathing index with the breathing parameter, which is detectable as the user is executing the breathing exercise, and determine a deviation therebetween; and
adapt the target breathing exercise based on the determined deviation.

45.-56. (canceled)
